(19) United States
(12) Patent Application Publication
(10) Pub. No.: US 2021/0337752 A1
(43) Pub. Date: Nov. 4, 2021

(54) PLANT TISSUE CULTURE DEVICES AND METHODS OF CULTURING AND HARVESTING PLANT SHOOT TIPS

(71) Applicant: Clemson University Research Foundation, Clemson, SC (US)

(72) Inventor: Jeffrey William Adelberg, Clemson, SC (US)

(21) Appl. No.: 17/271,246

(22) PCT Filed: Aug. 29, 2019
(86) PCT No.: PCT/US2019/048741

§ 371 Date: Feb. 25, 2021

Related U.S. Application Data
(60) Provisional application No. 62/724,410, filed on Aug. 29, 2018.

(51) Int. Cl. A01H 4/00; A01G 2/10

(52) U.S. Cl. CPC A01H 4/001; A01H 4/005; A01H 4/003; A01G 2/10

(57) ABSTRACT

The present disclosure describes a method of culturing and harvesting plant shoot tips. The method includes providing a sterile vessel configured to hold at least one plant with one or more root masses and a first set of shoot tips, cutting across a base of the shoot tips with the one or more root masses held in the vessel to cut a first plurality of cut shoot tips at a first time; then growing a second set of shoot tips from the one or more root masses in the vessel; and then cutting across the one or more root masses held in the vessel to cut a second plurality of cut shoot tips. Plant tissue culture devices are also described.

### RELATED APPLICATION(S)

The present application claims the benefit of and priority to U.S. Provisional Application Ser. No. ^{62/724,410}, filed Aug. 29, 2018, the contents of which are hereby incorporated by reference as if recited in full herein.

### FIELD

The present application is directed generally to plant micropropagation, and more particularly, to plant tissue culture devices and methods of culturing and harvesting plant shoot tips.

### BACKGROUND

The labor input per plant is one of the biggest factors in overall system efficiency of plant micropropagation because the worker time under the laminar flow hood is about two-thirds of the cost of growing a plant tissue culture. Prior studies of work flow in the laminar flow hood quantified the labor components of planting, cutting, and removing the plant. See, e.g., Alper, et al., Mass handling of watermelon microcuttings. ASAE. 37(4):1337-1343 (1994). In these prior studies, planting and cutting times were shown to be equal in affecting the total efficiency of the plant micropropagation system. Id. However, current vessels used in plant tissue cultures, such as a standard GA7 Magenta vessel 10 (see, e.g., FIG. 1), are not designed for multiple cuttings and the tools used for surgery and dissection (e.g., a scalpel 12 and forceps 14) only perform a single cut from a batch culture.

### SUMMARY

Embodiments of the present invention are directed to multiple-cut methods of culturing and harvesting plant shoot tips.

According to embodiments of the present invention, methods of culturing and harvesting plant shoot tips include providing a sterile vessel sized and configured to hold at least one plant comprising one or more root masses and a first set of shoot tips, cutting across a base of the first set of shoot tips with the one or more root masses held in the vessel to cut a first plurality of cut shoot tips at a first time, then growing a second set of shoot tips from the one or more root masses in the vessel, and then cutting across the base of the second set of shoot tips with the one or more root masses held in the vessel to cut a second plurality of cut shoot tips at a second time.

The second set of shoot tips may have an increased multiplication ratio providing more shoot tips than that the first set of shoot tips.

The cutting may be carried out using a single direction motion of a cutting tool above and across a base of the vessel.

The cutting may be carried out using a reciprocating motion of a cutting tool above and across a base of the vessel.

The cutting may be carried out using an electric knife.

The cutting may be carried out using a blade that is axially stationary and moved either automatically using a robotic arm or other electromechanical member or moved manually across and above the base.

The growing of the second set of shoot tips may include a growth period in a range of about 1 week to about 3 weeks, optionally comprising a growth period in a range of about 1.5 weeks to about 2.5 weeks.

The first plurality of cut shoot tips and the second plurality of cut shoot tips may be collected in a sterile receiver as microcuttings. The microcuttings may be placed in one or more different sterile vessels with nutrients in a greenhouse to grow into full grown plants.

The method of culturing and harvesting plant shoot tips may further include growing a third set of shoot tips from the one or more root masses held in the vessel after cutting the second set of shoot tips, and cutting across the base of the third set of shoot tips with the one or more root masses held in the vessel to cut a third plurality of cut shoot tips at a third time.

The method may further include growing a fourth set of shoot tips from the one or more root masses held in the vessel after cutting the third set of shoot tips, and then cutting across the base of the fourth set of shoot tips with the one or more root mass held in the vessel to cut a fourth plurality of cut shoot tips at a fourth time. At least the base of the vessel may remain sterile over each growing step allowing for several harvests of shoot tips from the same root masses, and optionally at least one of the second, third, and fourth set of shoot tips may have an increased number of shoot tips per plant relative to the first set of shoot tips.

The method may further include adding nutrients and/or water to the one or more root masses after a respective cutting. In some embodiments, the one or more root masses after the cutting of the first plurality of cut shoot tips may include a rooted matrix with one or more buds thereby allowing for rapid re-growth of one or more additional shoots to yield the second set of shoot tips.

The vessel can have a base releasably attached to a housing. The base may have a first height and the housing may have a second height. The second height of the housing can be about 2 times to about 10 times greater than the first height of the base. The base can hold the one or more root masses and can be sized and configured to allow the first and second set of shoot tips to grow a distance extending outwardly from the base and to be exposed when the housing is detached from the base.

The vessel may have a base with a height that is between about 0.5 inches and about 2 inches. The base may have a lateral width that is greater than the height. The base may hold the one or more root masses with the first and second set of shoot tips allowed to grow above the base.

The vessel can include a permeable substrate in the base configured to hold the root mass and comprising plant nutrients.

The base may hold a soilless substrate, and optionally a plant nutrient and moisture, along with the one or more root masses.

The base may be configured to hold the one or more root masses such that new shoot buds forming the second set of shoot tips after the cutting of the first plurality of cut shoot tips are allowed to grow above the height of the base during the growing step.

The housing may have a sidewall and/or a top with one or more permeable membranes.

The at least one plant may be a vascular plant.

The at least one plant may be a gymnosperm or an angiosperm.

The at least one plant may be a dicot or a monocot.

The at least one plant may be cannabis.

The at least one plant may be edible microgreens.

The at least one plant may be a potato.

The at least one plant may be a fruit tree or a timber tree.

The at least one plant may be an ornamental plant.

The vessel may include a base, and optionally an impermeable rigid or semi-rigid base, and a releasably attached housing. The base can be configured to hold the one or more root masses and the first and second sets of shoot tips can grow a distance extending outwardly from the base into the housing during the growing steps.

The vessel may further include a plant support member that is coupled to the base and that is configured to allow the first and second sets of shoot tips to grow through and above the plant support member. The support member may extend or reside at a height that defines a cut height for the cutting of the first plurality of cut shoot tips and second plurality of cut shoot tips. The base and/or the housing may optionally be visually transmissive.

The plant support member may be formed of thermoformed copolymer.

An edge of the plant support member may form an interference fit within the vessel.

The plant support member may be concave in shape and may apply a spring-like pressure on the shoot tips growing within the vessel such that the shoot tips cannot force the plant support member upward during growth.

The plant support member may have an open mesh or a grid configuration.

The mesh or grid configuration may include laterally spaced apart apertures that have an inverted funnel shape with a larger end residing further away from a bottom of the base to allow increased space for lateral expansion during plant growth.

The methods may further include removing the housing before the cutting steps and optionally rotating the base from a first orientation to a second orientation between each cutting step.

The cutting steps may be carried out to provide dozens of shoot tips as the first and second set of cut shoot tips.

The methods may further include reattaching the housing to the base for the growing of the second set of shoot tips.

The growing steps may be carried out in vitro in a sterile environment with the base of the vessel maintaining sterility during the growing of each of the first, second, third, and fourth sets of shoot tips.

The first plurality of cut shoot tips and the second plurality of cut shoot tips may have an increased rooting percentage and/or an increased percentage of survival in a greenhouse environment compared to at least one standard set of shoot tips collected individually using a scalpel from a corresponding root mass.

Further embodiments of the present invention are directed to a plant tissue culture device.

The plant tissue culture device may include a sterile base, optionally an impermeable rigid or semi-rigid base, and a sterile housing releasably attached to the sterile base. The base may have a first height and the housing may have a second height. The second height of the housing may be about 2 times to about 10 times greater than the first height of the base.

The base may be sized and configured to hold one or more plant root masses therein and may allow the one or more plant root masses to grow and produce a set of shoot tips with the set of shoot tips residing in the housing above a top edge of the base. The one or more root masses may optionally be held in a matrix of soilless nutrient material in the base.

The housing may include one or more permeable membranes.

The plant tissue culture device may further include a plant support member that is coupled to the base and configured to allow the set of shoot tips to grow through and above the plant support member. The plant support member may reside at a height that defines a cut height for cutting of the set of shoot tips.

The plant support member may be formed from a thermoformed copolymer.

An edge of the plant support member may form an interference fit with the base of the device.

The plant support member may be concave in shape and may apply a spring-like pressure on the shoot tips growing within the device such that the shoot tips cannot force the plant support member upward during growth.

The plant support member may have an open mesh or a grid configuration.

The mesh or grid configuration may include a plurality of laterally spaced apart apertures that have an inverted funnel shape with a larger end residing further away from a bottom of the base to allow increased space for lateral expansion during plant growth.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

### BRIEF DESCRIPTION OF THE DRAWINGS

FIG. 1 is a photograph of a prior art plant tissue culture device and cutting system.

FIG. 2 is a perspective view of a plant tissue culture device (or vessel) according to embodiments of the present invention.

FIG. 3A is a perspective view of the plant tissue culture device of FIG. 2 in use with the housing attached.

FIG. 3B is a perspective view of the plant tissue culture device of FIG. 3A with the housing removed.

FIG. 4 illustrates a set of shoot tips grown from one or more plant root masses and held in a permeable substrate according to embodiments of the present invention.

FIG. 5 is a perspective view of an exemplary cutting tool that may be used with the plant tissue culture device of FIG. 2.

FIG. 6 is a perspective view of another exemplary cutting tool that may be used with the plant tissue culture device of FIG. 2.

FIG. 7A is a perspective view of a plant tissue culture device with a plant support member (with the housing removed) according to embodiments of the present invention.

FIG. 7B is a perspective view of the plant tissue culture device of FIG. 7A with the housing attached.

FIG. 7C is side sectional schematic illustration of the plant tissue culture device of FIG. 7A with the plant support member having apertures with an inverted funnel shape according to embodiments of the present invention.

FIGS. 8A-8D illustrate a sequence of actions for the growing and cutting of multiple sets of shoot tips (T1-T4) using the plant tissue culture device of FIG. 2 according to embodiments of the present invention.

FIG. 8E is a flow chart of operations that may be used to carry out culturing and harvesting plant shoot tips according to embodiments of the present invention.

FIGS. 9A and 9B are photographs illustrating Petunia shoot tips grown in vitro using a prototype plant tissue culture device of the present invention. FIG. 9A illustrates the open device and FIG. 9B illustrates the closed device.

FIG. 10 is a chart showing the effect of the cutting cycles (2 weeks) and cutting type systems on the multiplication ratio of Petunia. The cutting type systems compared in FIG. 10 are a scalpel and forceps with agar media in a GA7 Magenta vessel (“C”) and an electric knife with Oasis® media in a plant tissue culture device of the present invention (“O”).

FIGS. 11A and 11B are charts showing the effect of the cutting cycles (2 weeks) and cutting type systems on the shoot length (cm) after 2 weeks in the greenhouse of Ragtime (FIG. 11A) and Suncatcher (FIG. 11B). The cutting type systems compared in FIG. 11A and FIG. 11B are a scalpel and forceps with agar media in a GA7 Magenta vessel (“C”) and an electric knife with Oasis® media in a plant tissue culture device of the present invention (“O”).

FIG. 12A is a graph of multiplication ratio versus cutting cycle (2 weeks cycle) showing the effect of the cutting cycles on the multiplication ratio of Ragtime. The cutting type systems compared in FIG. 12A are a scalpel and forceps with agar media in a GA7 Magenta vessel (“C”) and an electric knife with Oasis® media in a plant tissue culture device of the present invention (“O”).

FIG. 12B is a chart showing the effect of cutting cycles (2 weeks) and cutting type systems on the multiplication ratio of Ragtime. The cutting type systems compared in FIG. 12B are a scalpel and forceps with agar media in a GA7 Magenta vessel (“C”) and an electric knife with Oasis® media in a plant tissue culture device of the present invention (“O”).

FIG. 13 is a photograph illustrating shoot tips that were damaged when an electric knife (e.g., the exemplary cutting tool shown in FIG. 5) was used to cut the shoot tips after being grown in a prototype plant tissue culture device of the present invention.

FIG. 14A is a graph of shoot length (cm) versus cutting cycle (2 weeks cycle) showing the effect of cutting cycles and cutting type systems on the plant cutting rate per minute of Ragtime. The cutting type systems compared in FIG. 14A are a scalpel and forceps with agar media in a GA7 Magenta vessel (“C”) and an electric knife with Oasis® media in a plant tissue culture device of the present invention (“O”).

FIG. 14B is a chart showing the effect of the cutting cycles and cutting type systems on the plant cutting rate per minute of Ragtime. The cutting type systems compared in FIG. 14B are a scalpel and forceps with agar media in a GA7 Magenta vessel (“C”) and an electric knife with Oasis® media in a plant tissue culture device of the present invention (“O”).

FIG. 15A is a graph of shoot length (cm) versus cutting cycle (2 weeks cycle) showing the effect of the cutting cycles and cutting type systems on the shoot length (cm) after 2 weeks in the greenhouse of Ragtime. The cutting type systems compared in FIG. 15A are a scalpel and forceps with agar media in a GA7 Magenta vessel (“C”) and an electric knife with Oasis® media in a plant tissue culture device of the present invention (“O”).

FIG. 15B is a chart showing the effect of the cutting cycles and cutting type systems on the shoot length (cm) after 2 weeks in the greenhouse of Ragtime. The cutting type systems compared in FIG. 15B are a scalpel and forceps with agar media in a GA7 Magenta vessel (“C”) and an electric knife with Oasis® media in a plant tissue culture device of the present invention (“O”).

FIG. 16 is a chart showing the effects of cutting cycles and cutting systems on the multiplication ratio of Ragtime. The cutting systems compared in FIG. 16 are an electric knife with agar media in a plant tissue culture device of the present invention (“A”), a scalpel and forceps with agar media in a GA7 Magenta vessel (“C”), and an electric knife with Oasis® media in a plant tissue culture device of the present invention (“O”).

FIG. 17 is a chart showing the effects of three weeks cutting cycles and cutting systems on the plant cutting rate per minute of Ragtime. The cutting systems compared in FIG. 17 are an electric knife with agar media in a plant tissue culture device of the present invention (“A”), a scalpel and forceps with agar media in a GA7 Magenta vessel (“C”), and an electric knife with Oasis® media in a plant tissue culture device of the present invention (“O”).

FIG. 18 is a chart showing the effects of cutting cycles and cutting systems on the first three nodes length (cm) of Ragtime shoot. The cutting systems compared in FIG. 18 are an electric knife with agar media in a plant tissue culture device of the present invention (“A”), a scalpel and forceps with agar media in a GA7 Magenta vessel (“C”), and an electric knife with Oasis® media in a plant tissue culture device of the present invention (“O”).

FIG. 19 is a chart showing the effect of cutting systems and the effect of cutting cycles (cut per two weeks) on Petunia Ragtime multiplication ratio. The cutting systems compared in FIG. 19 are a scalpel and forceps with agar media in a GA7 Magenta vessel (“C”), a scalpel and forceps with agar media in a plant tissue culture device of the present invention (“KA”), a scalpel and forceps with Oasis® media in a plant tissue culture device of the present invention (“KO”), scissors with agar media in a plant tissue culture device of the present invention (“SA”), and scissors with Oasis® media in a plant tissue culture device of the present invention (“SO”).

FIG. 20 is a chart showing the effect of cutting systems and the effect of cutting cycles (cut per two weeks) on Petunia cutting rate per minute. The cutting systems compared in FIG. 20 are a scalpel and forceps with agar media in a GA7 Magenta vessel (“C”), a scalpel and forceps with agar media in a plant tissue culture device of the present invention (“KA”), a scalpel and forceps with Oasis® media in a plant tissue culture device of the present invention (“KO”), scissors with agar media in a plant tissue culture device of the present invention (“SA”), scissors with Oasis® media in a plant tissue culture device of the present invention (“SO”).

FIG. 21 is a chart showing the effect of cutting systems and the effect of cutting cycles (cut per two weeks) on Petunia Ragtime first three shoot nodes length (cm). The cutting systems compared in FIG. 21 are a scalpel and forceps with agar media in a GA7 Magenta vessel (“C”), a scalpel and forceps with agar media in a plant tissue culture device of the present invention (“KA”), a scalpel and forceps with Oasis® media in a plant tissue culture device of the present invention (“KO”), scissors with agar media in a plant tissue culture device of the present invention (“SA”), scissors with Oasis® media in a plant tissue culture device of the present invention (“SO”).

FIG. 22 is a chart showing the effect of cutting systems and the effect of cutting cycles (cut per two weeks) on Petunia Ragtime shoot tips fresh mass (g). The cutting systems compared in FIG. 22 are a scalpel and forceps with agar media in a GA7 Magenta vessel (“C”), a scalpel and forceps with agar media in a plant tissue culture device of the present invention (“KA”), a scalpel and forceps with Oasis® media in a plant tissue culture device of the present invention (“KO”), scissors with agar media in a plant tissue culture device of the present invention (“SA”), scissors with Oasis® media in a plant tissue culture device of the present invention (“SO”).

FIG. 23 is a photograph illustrating the growth of Ragtime after three weeks from the fourth cutting cycle. The photograph shows the difference in shoot and root systems in different media volume of Oasis® media (a) 160 mL, (b) 200 mL, (c) 240 mL, (d) 280 mL, and (e) 280 mL in agar media.

FIGS. 24A and 24B are photographs illustrating a grid on the top of Petunia shoot tips grown in vitro on Oasis® media (FIG. 24A) and agar media (FIG. 24B) after T1 cutting cycle in a plant tissue culture device according to embodiments of the present invention.

FIG. 25 is a photograph illustrating the cutting of Petunia shoot tips grown in vitro on Oasis® media in a base of the vessel by a blade and grid cutting system at T2 cutting cycle.

FIGS. 26A and 26B are graphs showing the effect of cutting cycle and medium type (FIG. 26A) and cutting type (FIG. 26B) on the multiplication ratio of Petunia Ragtime. The model had a fit of R²=0.811, P<0.0001.

FIG. 27 is a graph showing the effect of cutting cycles and medium type on the shoot fresh mass per cut (g) of Petunia Ragtime. The model had a fit of R²=0.534, P<0.0001.

FIG. 28 is a graph showing the effect of cutting cycles and medium type on the shoot node (cm) of Petunia Ragtime. The model had a fit of R²=0.764, P<0.0001.

FIG. 29 is a graph showing the effect of cutting cycles and medium type on the percentage of flowering tips of Petunia Ragtime. The model had a fit of R²=0.705, P<0.0001.

FIG. 30 is a graph showing the effect of cutting cycles and medium type on the percentage of hyperhydricity of Petunia Ragtime. The model had a fit of R²=0.709, P<0.0001.

FIGS. 31A and 31B are graphs showing the effect of cutting cycle on the water lost (mL) from a plant tissue culture device according to embodiments of the present invention without fed-batch treatment with respect to medium type and fed-batch treatment (FIG. 31A) and cutting types (FIG. 31B). The model had a fit of R²=0.984, P<0.0001.

FIGS. 32A and 32B are photographs showing Ragtime Petunia in Oasis® medium (FIG. 32A) and agar medium (FIG. 32B) in a prototype plant tissue culture device according to embodiments of the present invention before the cutting in the T4 cycle (8 weeks).

FIG. 32C is a photograph showing the Petunia after 14 weeks in the prototype plant tissue culture device shown in FIG. 32A and FIG. 32B in both media.

FIG. 33A is a bottom perspective view of an alternative plant support member according to embodiments of the present invention.

FIG. 33B is a side view of the plant support member of FIG. 33A.

FIG. 34A is a photograph of a prototype plant tissue culture device using the plant support member of FIG. 33A according to embodiments of the present invention.

FIG. 34B is a photograph illustrating Prunus cerassifera shoot tips grown in vitro using of the prototype plant tissue culture device of FIG. 34A.

FIG. 34C is a photograph illustrating the cutting of the Prunus cerassifera shoot tips shown in FIG. 34B using a scalpel and forceps.

### DETAILED DESCRIPTION

The present invention now is described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

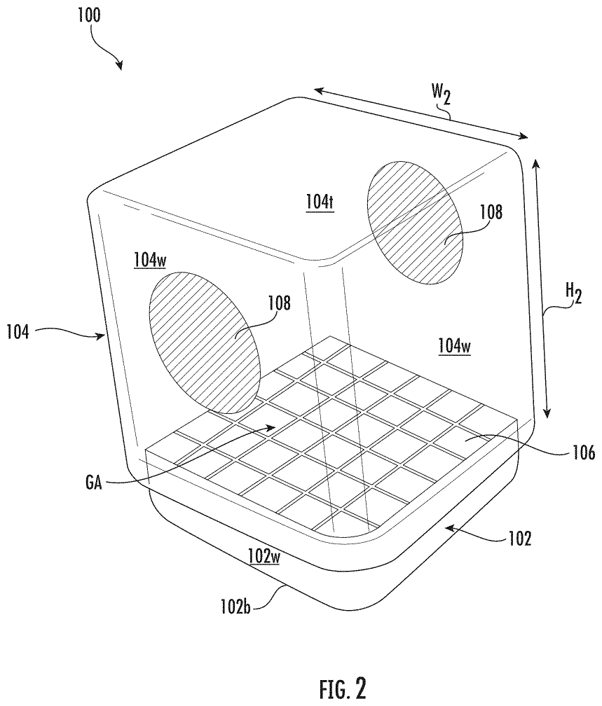

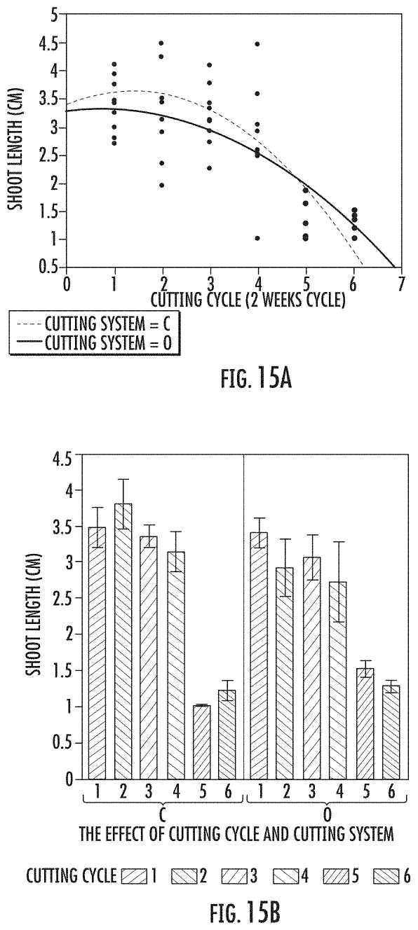

In general, embodiments of the present invention provide a plant tissue culture device that may be used to provide a vessel and culturing and harvesting tool to allow mechanized cutting of elongated plant shoot cuttings for plant micropropagation. Related methods of culturing and harvesting plant shoot tips are also provided. Embodiments of the present invention will now be discussed in greater detail with reference to FIGS. 2-32C.

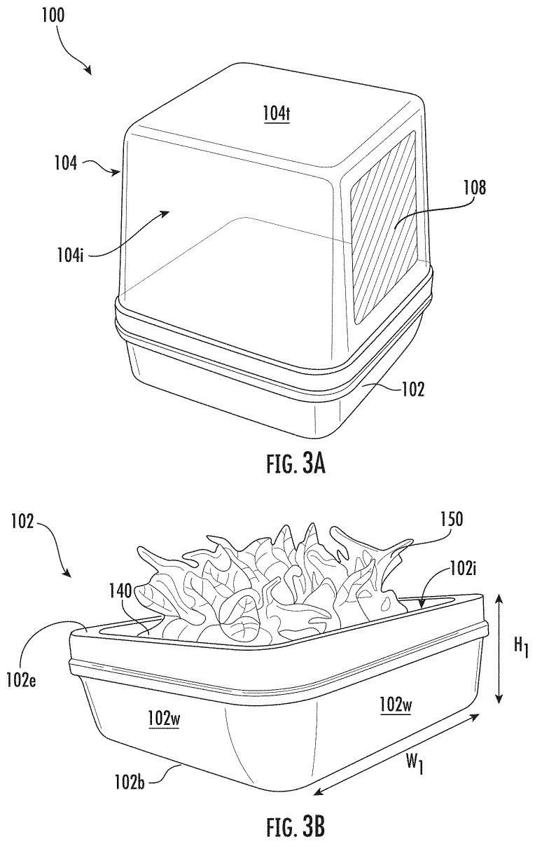

Referring to FIG. 2 and FIGS. 3A-3B, a plant tissue culture device 100 according to embodiments of the present invention is illustrated. The plant tissue culture device 100 (also interchangeably referred to herein as a “vessel” or a “smart vessel”) may comprise a base 102 and a housing 104. The base 102 of the device 100 may comprise a bottom 102b and one or more sidewalls 102w. The bottom 102b and the one or more sidewalls 102w enclosure/surround an interior chamber 102i of the base 102. The housing 104 of the device 100 may comprise a top (or ceiling) 104t and one or more sidewalls 104w. The top 104t and the one or more sidewalls 104w enclosure/surround an interior chamber 104i of the housing 104. The housing 104 can be releasably attached to the base 102. Together, the interior chambers 102i, 104i of the base 102 and the housing 104 provide a “growing area” (GA) within the plant tissue culture device 100.

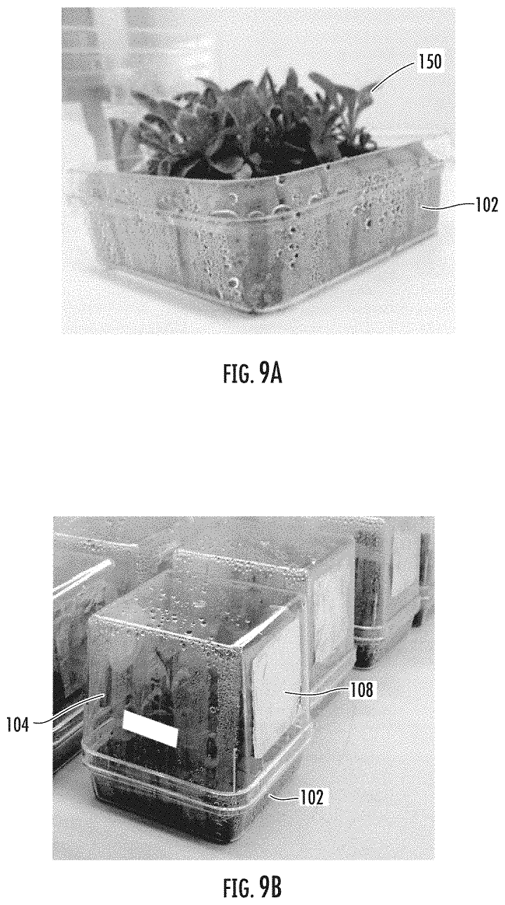

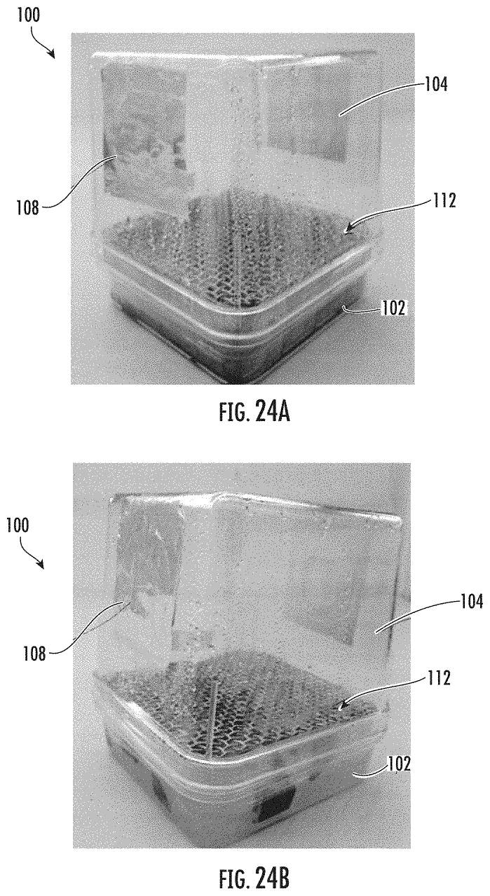

As shown in FIG. 2 and FIG. 3B, the base 102 has a first height H₁ and the housing 104 has a second height H₂. In some embodiments, the height H₂ of the housing 104 may be about 2 times to about 10 times greater than the height H₁ of the base 102. For example, in some embodiments, the height H₁ of the base 102 may be in a range of the about 0.5 inches to about 2 inches and the height H₂ of the housing 104 may be in a range of about 1 inch to about 20 inches. As will be discussed in further detail below, the additional height H₂ of the housing 104 provides space within the interior chamber 104i of the housing 104 to allow plant shoot tips 150 to grow (i.e., within the growing area GA) (see also, e.g., FIG. 3A). In some embodiments, the base 102 may have a lateral width W₁ that is greater than its height H₁. The housing 104 may also have a lateral width W₂ that is about equal to the lateral width W₁ of the base 102 such that the housing 104 may be releasably attached and secured to the base 102. Optionally, in some embodiments, the base 102 and/or housing 104 may be visually transmissive (see, e.g., FIGS. 9A-9B and FIGS. 24A-24B).

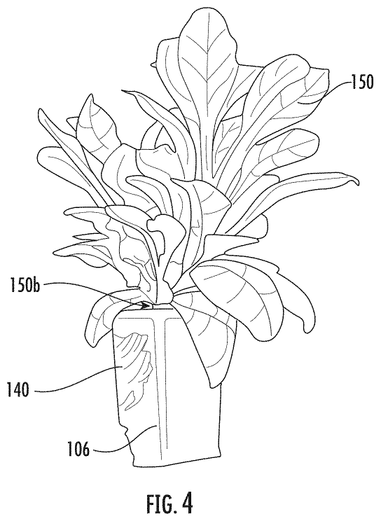

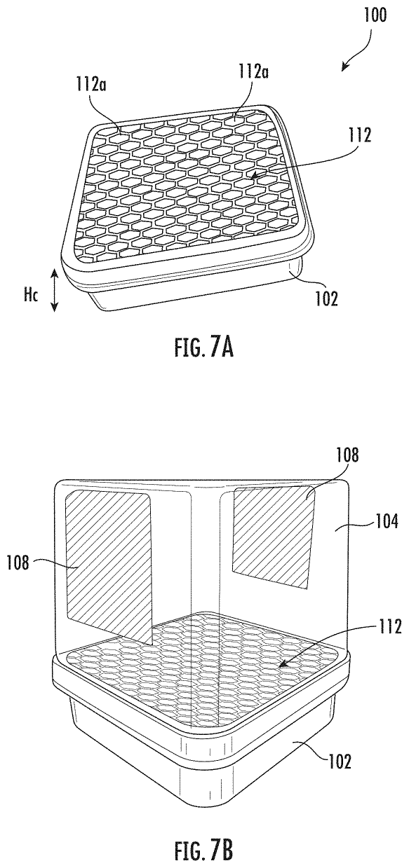

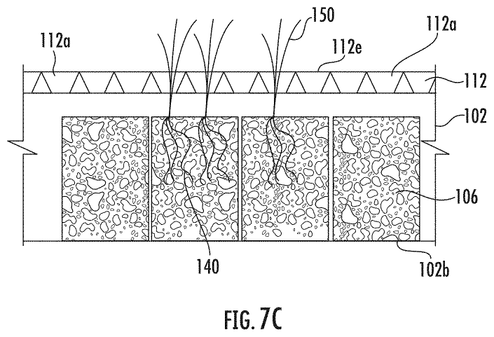

In some embodiments, both the base 102 and the housing 104 of the plant tissue culture device 100 are sterile. As used herein, “sterile” means free from contamination by other organisms, such as, for example, bacteria, insects, fungi, viruses, and weeds. Optionally, in some embodiments, the base 102 may be an impermeable rigid or semi-rigid, self-supporting base 102. As used herein, the term “rigid” means that the base 102 is unable to bend or be forced out of shape, i.e., not flexible. As used herein, the term “semi-rigid” means that the base 102 has a self-supporting shape, but may flex when sufficient force is applied. As used herein, the term “self-supporting” means that the base 102 will retain a pre-formed shape when apart from the housing 104 and/or when holding a plant root mass or masses 140 therein (see, e.g., FIG. 4 and FIG. 7C).

In some embodiments, the housing 104 may comprise at least one permeable membrane 108. In some embodiments, the at least one permeable membrane 108 may be used for ventilation. The at least one permeable membrane 108 may allow for gases, such as, oxygen, nitrogen, and carbon dioxide to enter and exit the device 100 and/or to allow water evaporation. For example, as shown in FIG. 2, FIG. 3A, and FIG. 7B, in some embodiments, one or more sidewalls 104w and/or the top 104t of the housing 104 may comprise one or more permeable membranes 108.

In some embodiments, the membrane(s) 108 may be covered by a removable cover 108c to reduce water evaporation from the device 100. For example, as shown in FIG. 9B, in some embodiments, the at least one membrane 108 may be covered with aluminum foil or other suitable material.

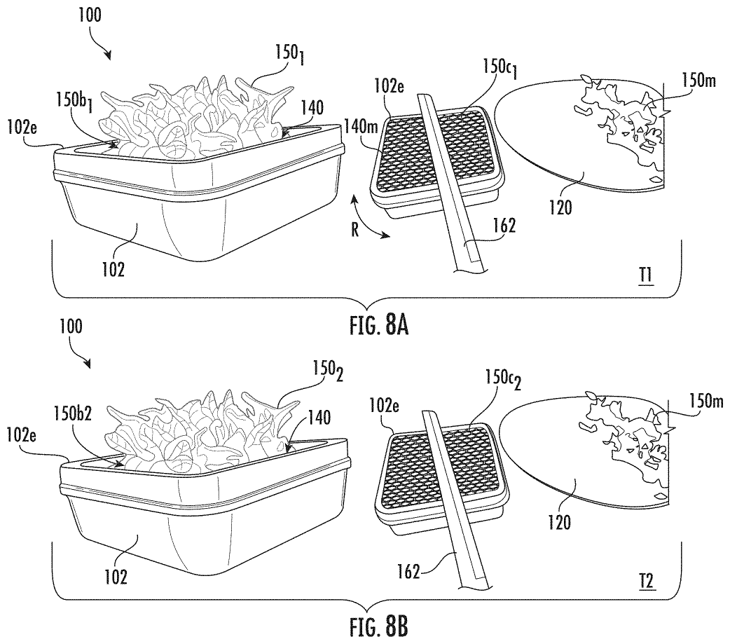

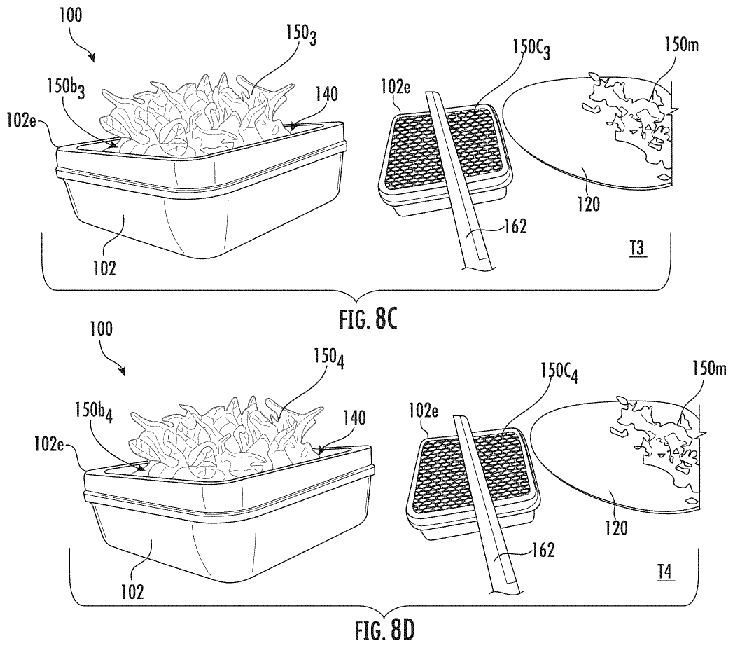

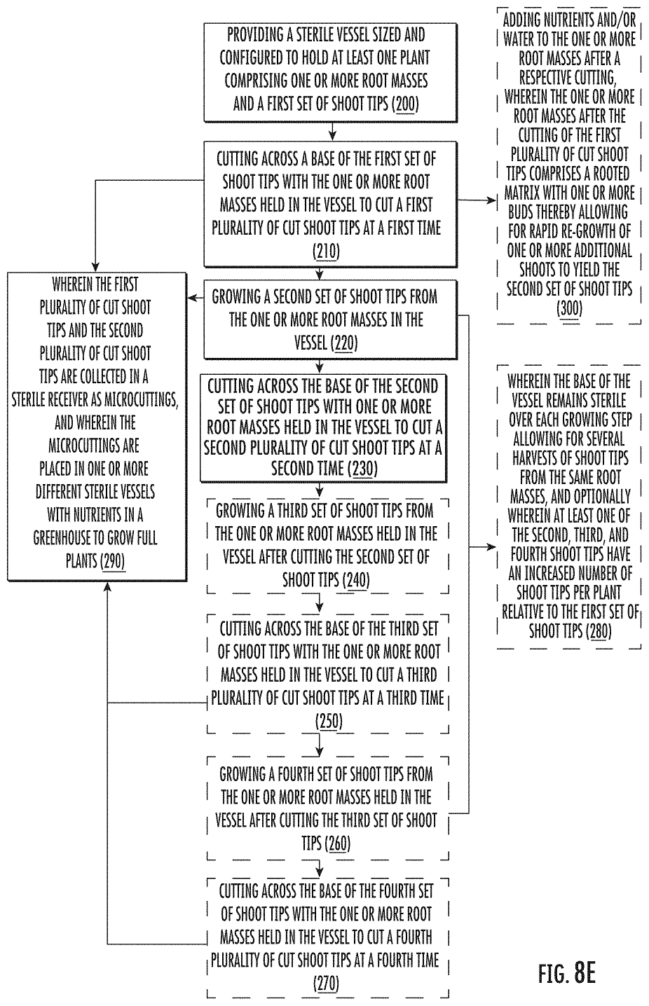

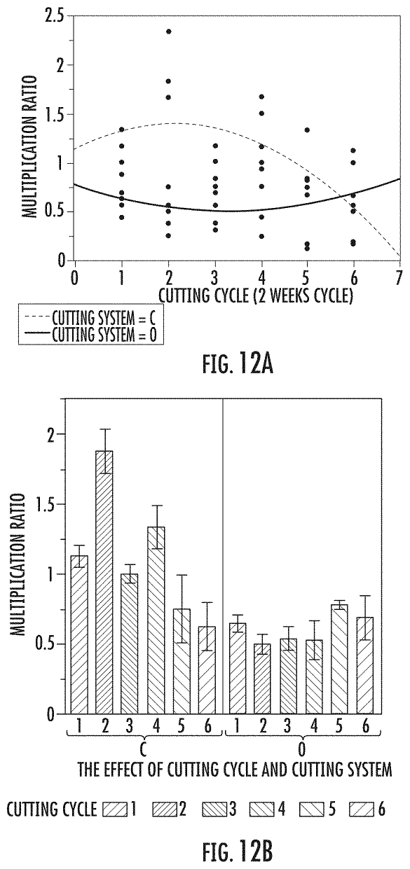

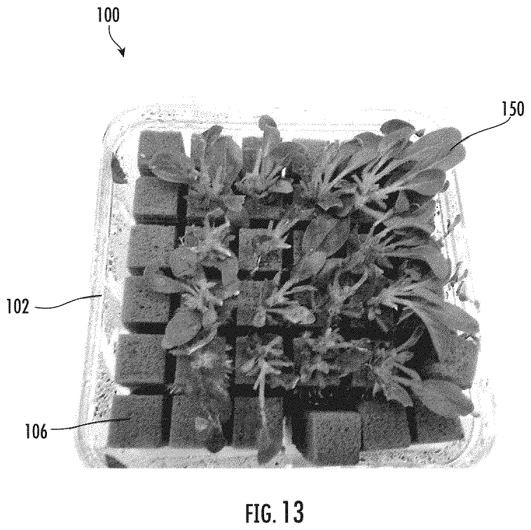

As shown in FIG. 3B and FIG. 4, in some embodiments, the base 102 of the plant tissue culture device 100 is sized and configured to hold one or more plant root masses 140 therein. As will be discussed in further detail below, the base 102 allows the one or more plant root masses 140 to grow and produce shoot tips 150 within the device 100 (see also, e.g., FIG. 7C, FIGS. 8A-8D, FIGS. 9A-9B, FIG. 13, and FIGS. 32A-32C). As shown in FIG. 2, FIG. 4, and FIG. 7C, in some embodiments, the device 100 may comprise a permeable substrate 106 in the base 102 (see also, e.g., FIG. 12). The permeable substrate 106 may be configured to hold the one or more plant root masses 140. In some embodiments, the permeable substrate 106 may comprise plant nutrients. In some embodiments, the permeable substrate 106 may comprise a matrix of soilless nutrient material. For example, in some embodiments, along with the one or more root masses 140, the base 102 may be configured to hold the permeable substrate 106 (e.g., the soilless nutrient material) with a plant nutrient and moisture. An example of a matrix of soilless nutrient material is Oasis® media (Grower Solutions, Kent, Ohio, USA).

As shown in FIGS. 3A-3B, in some embodiments, as a set of shoot tips 150 grow from the one or more plant root masses 140, the shoot tips 150 reside within the housing 104 and grow a distance (D) extending outwardly from (i.e., above) the base 102 of the device 100 (see also, e.g., FIG. 7C). The base 102 is sized and configured to allow the sets of shoot tips 150 to grow out from the base 102 and into the interior chamber 104i of the housing 104 (i.e., above a top edge 102e of the base 102 when the device 100 is used in the orientation shown in FIGS. 3A-3B) such that the shoot tips 150 may be exposed when the housing 104 is detached and removed from the base 102 (FIG. 3B). As will be discussed in further detail below, after a pre-determined growth period (e.g., in some embodiments, between about 1 week and about 3 weeks), a first set of shoot tips 150₁ extending (growing) above the top edge 102e of the base 102 may be cut (i.e., a first plurality of cut shoot tips 150c₁) (see, e.g., FIG. 8A). The base 102 may be configured to continue to hold the one or more root masses 140 such that new shoot buds (which will form a second set of shoot tips 150₂) are allowed to grow out from the base 102 (or above the top edge 102e of the base 102) (i.e., after the cutting of the first set of shoot tips 150₁) (see, e.g., FIG. 8B). The plant tissue culture device 100 of the present invention is configured to allow for multiple cuttings and re-growth of successive different sets of shoot tips (e.g., 150₁, 150₂, 150₃, 150₄ . . . 150ₙ) (typically, n=2 to 100) (see, e.g., FIGS. 8A-8D).

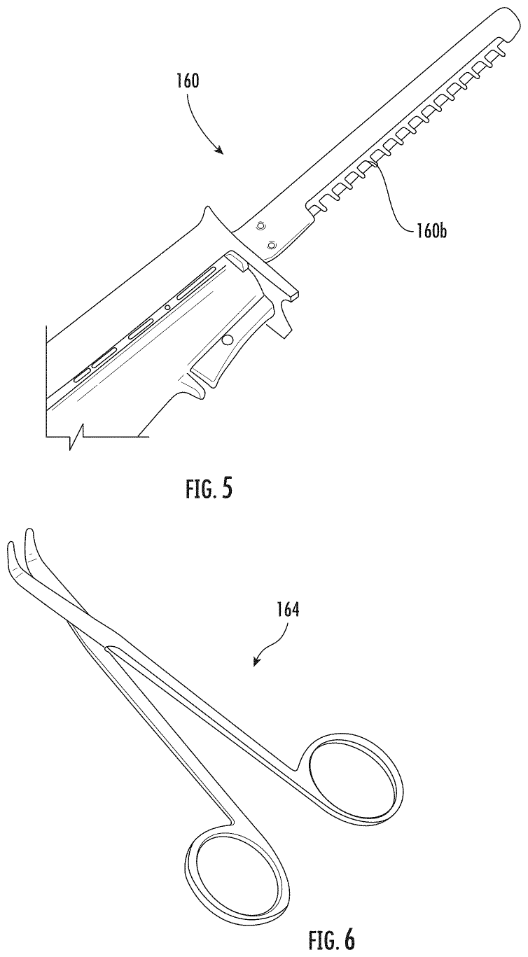

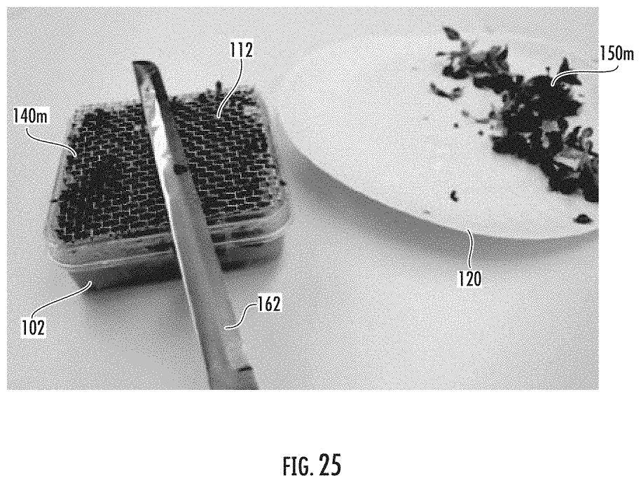

In some embodiments, each set of shoot tips 150₁, 150₂ may be cut using a cutting tool 160. As shown in FIG. 5, in some embodiments, the cutting tool 160 may be an electric knife having one or more long serrated blades 160b that, when powered on, continuously move lengthways to provide a sawing/cutting action. In some embodiments, the cutting tool 160 can comprise an elongated blade 162 that may be used to cut each set of shoot tips 150₁, 150₂ (see, e.g., FIGS. 8A-8D and FIG. 25). In some embodiments, the blade 162 may be axially stationary and moved automatically using a robotic arm or other electromechanical member (not shown) to cut the shoot tips 150₁, 150₂. As shown in FIG. 6, in some embodiments, the cutting tool 160 can comprise a pair of scissors 164 that may be used to cut each set of shoot tips 150₁, 150₂.

Referring to FIGS. 7A-7C, in some embodiments, the plant tissue culture device 100 may further comprise a plant support member 112 coupled to the base 102. The plant support member 112 can be configured to allow the shoot tips 150 to grow through and above or beyond the plant support member 112 (and the base 102). In some embodiments, the plant support member 112 resides at a height that defines a cut height H_{C} for the cutting of a set of shoot tips 150. In some embodiments, the cut height H_{C} may be equal the height H₁ of the base 102. In some embodiments, the cut height H_{C} may be greater than the height H₁ of the base 102.

As shown in FIGS. 7A-7C, in some embodiments, the plant support member 112 has an open mesh or a grid configuration. In some embodiments, the mesh or grid configuration of the plant support member 112 may comprise a plurality of laterally spaced apart apertures 112a. As shown in FIG. 7C, in some embodiments, the apertures 112a may have an inverted funnel shape with a larger end 112e residing further away from the bottom 102b of the base 102. The inverted funnel shape of the apertures 112a may allow for increased space for lateral expansion during plant growth.

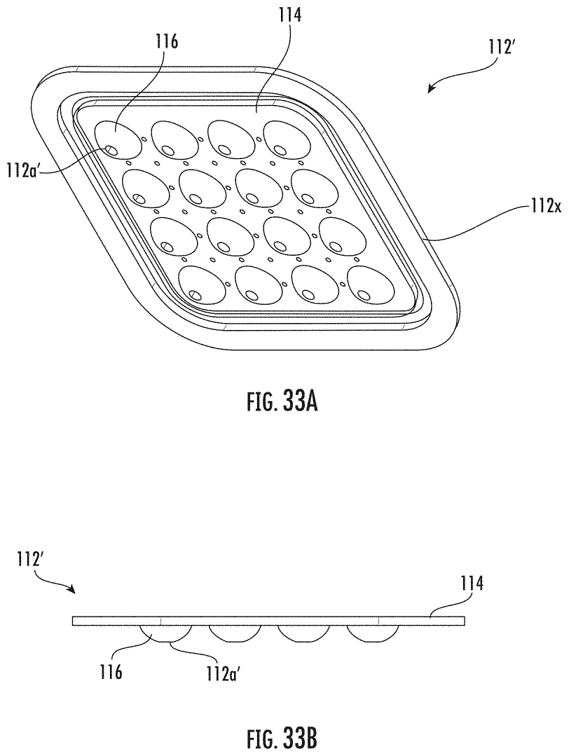

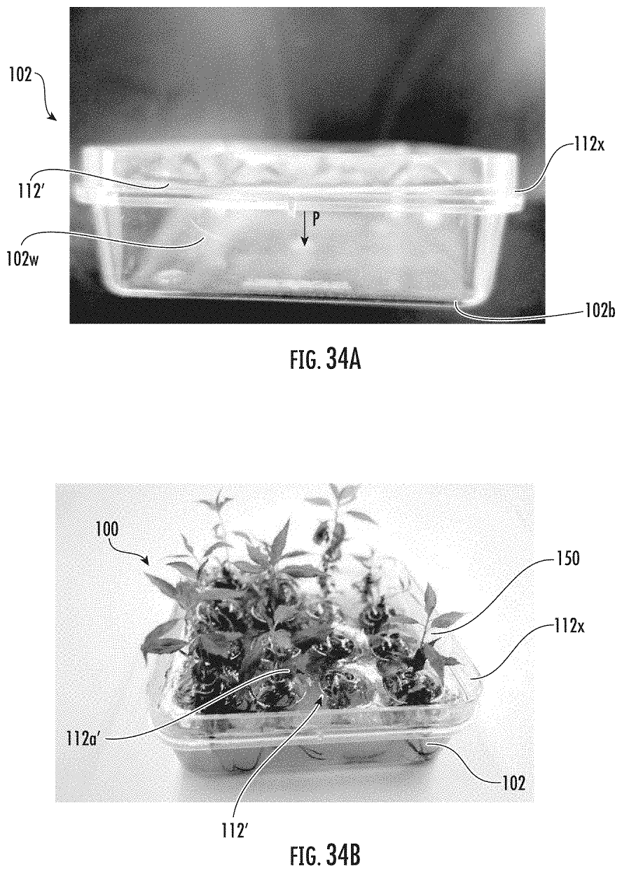

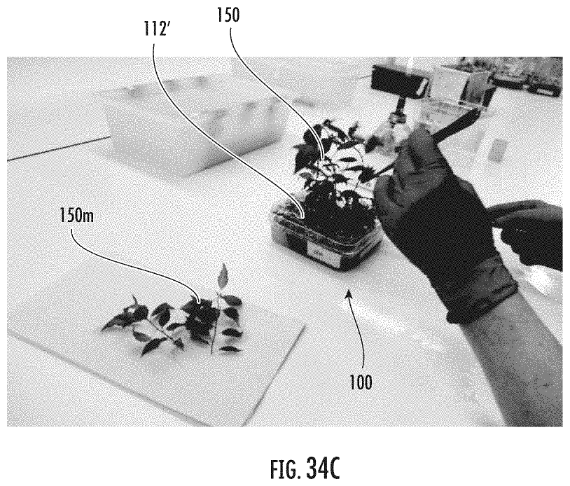

An alternative plant support member 112′ according to embodiments of the present invention in illustrated in FIGS. 33A and 33B. In some embodiments, the plant support member 112′ may comprise a base member 114 and a plurality of recesses 116. As shown in FIG. 33B, the plurality of recesses 116 may be concave in shape. Similar to the open mesh or grid configuration, the plant support member 112′ may comprise apertures 112a′ that allow the shoot tips 150 to grow through and above or beyond the plant support member 112′ (and the base 102) of the plant tissue culture device 100 (see also, e.g., FIGS. 34B and 34C). In some embodiments, the apertures 112a′ may reside within each recess 116. In some embodiments, apertures 112a′ may reside in the base 114 of the plant support member 112′ between the recesses 116. Similar to the inverted funnel shape of apertures 112a discuss above, the concave shape of the recesses 116 may allow for increased space for lateral expansion during plant growth.

Referring to FIGS. 34A and 34B, in some embodiments, the edge 112x of the plant support member 112′ may form an interference fit within the plant tissue culture device 100 (e.g., with the base 102 of the device 100). As shown in FIG. 34A-34C, in some embodiments, the plant support member 112′ may be concave in shape (see, e.g., FIG. 34A). The concave shape of the plant support member 112′ may apply a spring-like pressure P on the shoot tips 150 growing within the device 100 such that the shoot tips 150 cannot force the plant support member 112′ upward during growth. In some embodiments, the plant support member 112′ may comprise a thermoformed copolymer. For example, in some embodiments, the plant support member 112′ may be formed from polycarbonate.

Methods of culturing and harvesting plant shoot tips 150 are also provided by embodiments of the invention. FIG. 8E is a flow chart of operations that may be used to carry out culturing and harvesting plant shoot tips according to embodiments of the present invention. In some embodiments, a method of culturing and harvesting plant shoot tips 150 may comprise (i) providing a sterile vessel (i.e., a plant tissue culture device) 100 configured to hold at least one plant comprising one or more root masses 140 and a first set of shoot tips 150₁ (block 200), (ii) cutting across a base 150b₁ of the first set of shoot tips 150₁ with the one or more root masses 140 held in the vessel 100 to cut a first plurality of cut shoot tips 150c₁ at a first time (T1) (block 210); then (iii) growing a second set of shoot tips 150₂ from the one or more root masses 140 while remaining in the vessel 100 (block 220); and then (iv) cutting across the base 150b₂ of the second set of shoot tips 150₂ with the one or more root masses 140 held in the vessel 100 to cut a second plurality of cut shoot tips 150c₂ at a second time (T2) (block 230) (see also, e.g., FIG. 8A and FIG. 8B).

According to embodiments of the present invention, the growing step(s) may be carried out in vitro in a sterile environment with at least the base 102 of the vessel 100 maintaining sterility during the growing of the first and second set of shoot tips 150₁, 150₂.

According to some embodiments of the present invention, the cutting step(s) may provide a multitude (dozens) of new shoot tips 150 from the first plurality and the second plurality of cut shoot tips 150c₁, 150c₂. As shown in FIGS. 8A-8D and FIG. 25, in some embodiments, the cutting of the shoot tips 150₁, 150₂ may be carried out using a single direction motion of a cutting tool (e.g., an elongate blade 162) moved above and across the top edge 102e of the base 102 of the vessel 100 (i.e., the cut height H_{C}). In some embodiments, the cutting of the shoot tips 150₁, 150₂ may be carried out using a reciprocating motion of a cutting tool (e.g., an elongate blade 162) above and across the top edge 102e of the base 102 of the vessel 100. In some embodiments, the blade 162 may be axially stationary and moved automatically using a robotic arm or other electromechanical member (not shown) to cut the shoot tips 150₁, 150₂. In some embodiments, the blade 162 may be moved manually across and above the base 102 of the vessel 100 to cut the shoot tips 150₁, 150₂. In some embodiments, the cutting of the shoot tips 150₁, 150₂ may be carried out using an electric knife 160 (see, e.g., FIG. 5).

In some embodiments, the methods may further comprise removing the housing 104 of the vessel 100 before performing the cutting step(s) (see, e.g., FIGS. 8A-8D and FIG. 25). Optionally, methods may comprise rotating the base 102 from a first orientation to a second orientation (R) before performing at least one of the cutting steps (FIG. 8A). For example, in some embodiments, the methods may comprise performing a cutting step in a first direction across the top edge 102e of the base 102 of the vessel 100, then rotating the base 102, 90 degrees or 180 degrees, then performing a second cutting in the same direction across the top edge 102e of the base 102. In some embodiments, methods of the present invention may further comprise reattaching the housing 104 to the base 102 of the vessel 100 for growing the second set of shoot tips 150₂ (see, e.g., FIG. 8B).

In some embodiments, methods of the present invention may further comprise adding nutrients and/or water to the one or more root masses 140 after a respective cutting (block 300). In some embodiments, the one or more root masses 140 after the cutting of the first set of shoot tips 150₁ may comprise a rooted matrix 140m with one or more buds within the interior chamber 102i of the base 102 (FIGS. 8A-8D and FIG. 25). This rooted matrix 140m may allow for the rapid re-growth of one or more additional shoots 150 to yield the second set of shoot tips 150₂.

In some embodiments, as each of the first and second sets of shoot tips 150₁, 150₂ are cut, the respective first plurality and second plurality of cut shoot tips 150c₁, 150c₂ may be collected in a sterile receiver 120 as microcuttings 150m (block 290) (see, e.g., FIGS. 8A-8D and FIG. 25). In some embodiments, these microcuttings 150m can be placed into one or more separate new/different sterile vessels 100 with nutrients in a greenhouse where the microcutting 150m are grown to produce roots, eventually, growing into full grown plants (block 290).

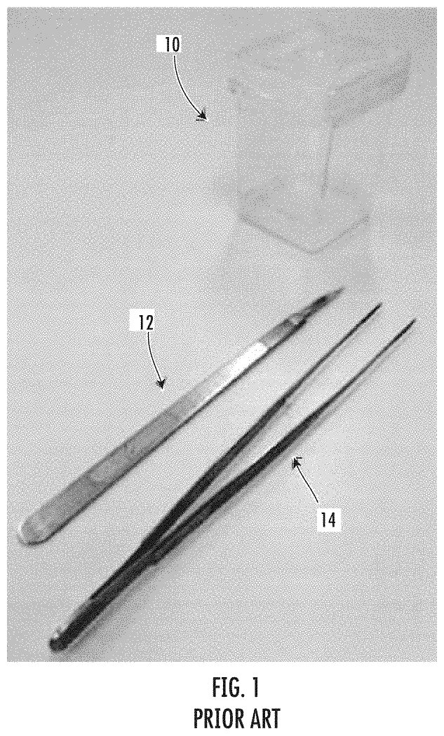

In some embodiments, the second set of shoot tips 150₂ has an increased multiplication ratio providing more shoot tips 150 than that of the first set of shoot tips 150₁. According to embodiments of the present invention, the first plurality of cut shoot tips 150c₁ and the second plurality of cut shoot tips 150c₂ may have an increased rooting percentage and/or an increased percentage of survival in a greenhouse environment compared to at least one standard set of shoot tips 150 collected individually using a scalpel from a corresponding root mass 140 (i.e., single cut systems, for example, shown in FIG. 1).

In some embodiments, the growing step for the second set of shoot tips 150₂ may comprise a growth period (e.g., T2) in a range of about 1 week to about 3 weeks. Optionally, the growing step for the second set of shoot tips 150₂ may comprise a growth period (e.g., T2) in a range of about 1.5 weeks to about 2.5 weeks.

In some embodiments, methods of the present invention may further comprise growing a third set of shoot tips 150₃ from the one or more root masses 140 held in the vessel 100 after cutting the second set of shoot tips 150₂ (block 240) (see also, e.g., FIG. 8C). Similar to the first and second sets of shoot tips 150₁, 150₂, after a growth period (e.g., about 1 week to about 3 weeks), the third set of shoot tips 150₃ may then be cut across the base 150b₃ of the third set of shoot tips 150₃ with the one or more root masses 140 held in the vessel 100 to cut a third plurality of cut shoot tips 150c₃ at a third time (T3) (block 250). Like the plurality of the second set of shoot tips 150c₂, the plurality of the third plurality of cut shoot tips 150c₃ may be collected as microcuttings 150m and placed in a greenhouse for further growth (block 290).

In some embodiments, methods of the present invention may further comprise growing a fourth set of shoot tips 150₄ from the one or more root masses 140 held in the vessel 100 after cutting the third set of shoot tips 150₃ (block 260) (see also, e.g., FIG. 8D). Similar to the prior cutting steps, after a growth period, the fourth set of shoot tips 150₄ may then be cut across the base 150b₄ of the fourth set of shoot tips 150₄ with the one or more root masses 140 held in the vessel 100 to cut a fourth plurality of cut shoot tips 150c₄ at a fourth time (T4) (block 270) (and collected as microcuttings 150m (block 290)). Each growth period (i.e., T1, T2, T3, T4) can be the same duration or can be different in duration.

According to embodiments of the present invention, the base 102 of the vessel 100 can remain sterile over each growing step and may allow for several harvests of shoot tips (150₁ to 150ₙ) from the same root mass 140. In some embodiments, at least one of the second, third, or fourth set of shoot tips 150₂, 150₃, 150₄ may have an increased number of shoot tips 150 per plant relative to the first set of shoot tips 150₁. The same housing 104 of a different housing 104 can be used for growing each subsequent set of shoot tips 150₂, 150₃, 150₄. The housing 104 can remain sterile over each growing step.

Devices and methods of the present invention may be suitable for use with a variety of different plants. Exemplary types of plants that may be suitable for use include, but are not limited to, a vascular plant, a gymnosperm or an angiosperm, a dicot or a monocot, cannabis, edible microgreens, a potato, a fruit tree or a timber tree, or an ornamental plant.

The present subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### EXAMPLES

The following EXAMPLES provide illustrative embodiments. Certain aspects of the following EXAMPLES are disclosed in terms of techniques and procedures found or contemplated by the present inventor to work well in the practice of the embodiments. In light of the present disclosure and the general level of skill in the art, those of skill will appreciate that the following EXAMPLES are intended to be exemplary only and that numerous changes, modifications, and alterations can be employed without departing from the scope of the presently claimed subject matter.

Several experiments were performed to investigate the effects of processes on worker efficiency under the laminar flow hood, and the quality of Petunia shoot tips in lab and the greenhouse. The multiple-cut system utilized the plant tissue culture device 100 described above (FIGS. 2 and 3A-3B) (referred to below as “the vessel” or “the ‘smart’ vessel”), Oasis® media (i.e., a permeable soilless substrate 106) (Grower Solutions, Kent, Ohio, USA), and an electric knife 160 (FIG. 5) that allowed the cutting of many shoot tips 150 at once. The cutting was repeated on the same vessel periodically, with regrowth coming from the stool shoot.

The first experiment (Example 1) tested the effect of a scalpel and forceps cutting system and using agar media with a GA7 Magenta vessel (Magenta Corp., Chicago, Ill., USA) in a single batch cycle standard control (e.g., FIG. 1), or with repeated cutting of stool shoots (2 weeks), and an electric knife multiple-cut system growing in Oasis® media with the “smart” vessel, two cutting cycles, and fed-batch technique on Petunia genotypes (Ragtime and Suncatcher). The second experiment (Example 2) was conducted to investigate the effect of long-term culture of six cutting cycles (2 weeks), and fed-batch techniques on Petunia Ragtime shoot culture in a lab and the subsequent plant quality in a greenhouse. The third experiment (Example 3) was conducted to compare the scalpel and forceps cutting system using agar media with a GA7 Magenta vessel with an electric knife multiple-cut system in the “smart” vessel with agar media and Oasis® media of four cutting cycles (3 weeks). The effect of media volume in Oasis® media was tested. The fourth experiment (Example 4) was performed to compare the scalpel and forceps cutting system using agar media with a GA7 Magenta vessel with two cutting tools: the scalpel and forceps and scissors in the “smart” vessel using agar media and Oasis® media for four cutting cycles (2 weeks). The effect of media volume in Oasis® media was tested. An experiment (Example 5) was also performed to compare the cutting types (blade and grid system vs. scalpel and forceps system) in two different media, agar and Oasis® media.

### Example 1: Two Cycles of Multiple Cutting on Agar and Oasis® with Scalpel and Forceps and Electric Knife Using Two Genotypes of Petunia

The first experiment aimed to screen the effects of Petunia genotypes Ragtime and Suncatcher, Oasis® media (Grower Solutions, Kent, Ohio, USA) in the “smart” vessel, cutting cycles, and fed-batch technique on Petunia microcutting and the microcutting quality in the greenhouse.

### Methods and Materials

Plant material: Petunia x hybrida (3832 Ragtime and 3859 Suncatcher) stage II tissue cultures were obtained from (Ball Horticultural Co., West Chicago, Ill., USA). Stock plantlets were grown on Basal MS medium (Murashige and Skoog, 1962) supplemented with 0.01% w/v thiamine HCl, 0.05% w/v nicotinic acid, 0.05% w/v pyridoxine HCl, 10.0% w/v myo-inositol, 0.2% w/v glycine, 0.65% w/v agargel (agar and phytagel, Sigma A3301), 0.5% v/v iron sulfate EDTA, and 3% w/v sucrose. Medium pH was adjusted to 5.8. Media are autoclaved in vessels at 121° C. for 25 minutes.

In vitro micropropagation: Sixteen shoot tips grown on Oasis® media in a “smart” vessel (FIGS. 9A and 9B) with 160 mL MS medium (Murashige and Skoog, 1962) supplemented with 0.01% w/v thiamine HCl, 0.05% w/v nicotinic acid, 0.05% w/v pyridoxine HCl, 10.0% w/v myo-inositol, 0.2% w/v glycine, 0.5% v/v iron sulfate EDTA, and 3% w/v sucrose. Medium pH was adjusted to 5.8. The ventilation membranes of the “smart” vessel were covered with Aluminum foil to reduced water evaporation (FIG. 9B). Plants were cultured at 23±2° C. and cool white fluorescent light (30 μmol m⁻²s⁻¹). Six shoot tips grown on MS medium supplemented with 0.65% w/v agargel as described above in a GA7 Magenta vessel (Magenta Corp., Chicago, Ill., USA) that was considered as a control (standard vessel, agar, scalpel and forceps).

Cutting cycles and fed-batch techniques: The shoot tips in the “smart” vessel were cut every two weeks with electric knife (FIG. 5) while recording the cutting time. The control shoot tips in the GA7 Magenta vessel were cut with scalpel and forceps (FIG. 1). Following the stool cut, fed-batch techniques (water and medium) were applied on Oasis® culture by reset the volume of 160 mL with sterile water or medium and compared with Oasis® batch culture.

Greenhouse acclimatization: Non-rooted shoot tips T1 and T2 were transferred directly to the soilless mix (Fafard 3B) where six shoot tips from each vessel transferred to six cells in the 1206 pack. Plants grew for 2 weeks under mist frequency of 8 sec every 10 min during the daylight hours (latitude=34.67350, and longitude=−82.8326) in Fafard 3B soilless mix (Canadian sphagnum peat moss, 7.62/20.32 cm processed pine bark, perlite, vermiculite, wetting agent, starter fertilizer and dolomitic limestone; Sun Gro Horticulture, Agawam, Mass., USA).

Measurements: Multiplication ratio of T1 and T2 and plant cutting rate per min were counted in the laboratory. Survival %, % rooted, shoots length (cm), and numbers of leaves were counted in the greenhouse.

Multiplication ratio=the number microcuttings/initial shoot tips in the vessel

Plant cutting rate per min=the multiplication ratio/the cutting time (min)

The system efficiency was calculated by using the equation below:

System efficiency=the sum of multiplication ratio for each cutting cycle×the average of plant cutting rate of the cutting cycles.

Experimental design and data analysis: A complete random design (2×2×2×3 full factorial) was conducted with replicates of the treatments. The factorial model terms were considered significant at P<0.05 (Table 3). The experiment design, analysis, and graphs were created using JMP version 12.0 (SAS Inst., Cary, N.C., USA).

### Results

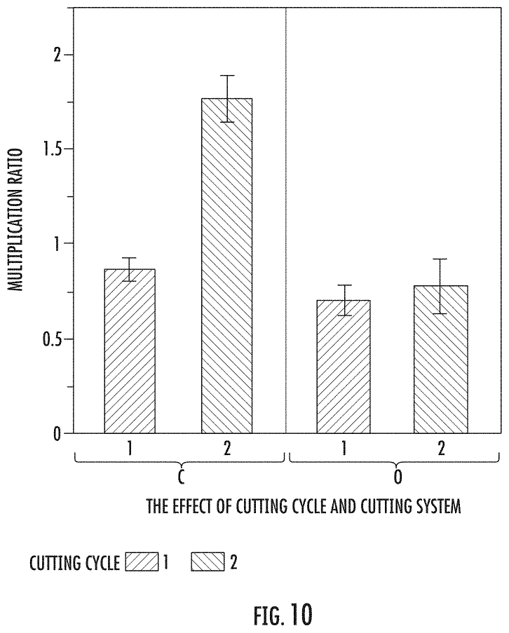

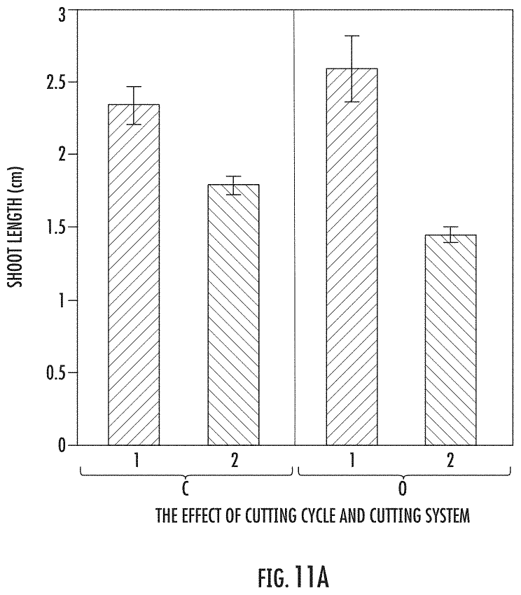

Labor components in the laminar flow hood: Plant multiplication, with the scalpel and forceps in agar and GA7 Magenta vessel system, Petunia T1 was 0.86±0.5 fold and T2 was 1.76±0.5 fold (FIG. 10). In the electric knife multiple-cut system, T1 was 0.7±0.5 fold and T2 was 0.76±0.5 fold. The second stool shoot cut T2 had more shoot tips than T1 in both cutting systems. Petunia Genotypes and fed-batch techniques did not affect the multiplication ratio. Plant cutting rate of scalpel and forceps was 1.25±0.1 min/vessel to cut 6 shoot tips in GA7 Magenta vessels, and electric knife used 0.76±0.1 min/vessel to cut 16 shoots in the “smart” vessel. Plant cutting rate per min for the scalpel and forceps in agar and GA7 Magenta vessel system was 0.96±0.3 min⁻¹ for T1 and 1.0±0.3 min⁻¹ for T2. The electric knife multiple-cut system had cutting rate of 0.96±0.3 min⁻¹ for T1 and 1.1±0.3 min⁻¹ for T2.

System Efficiency: After two cutting cycles, the total efficiency of scalpel and forceps in agar and GA7 Magenta vessel system and electric knife multiple-cut in Oasis® media and the “smart” vessel system were calculated by using the numbers from the previous paragraphs and the equation below:

System Efficiency of scalpel and forceps in agar and GA7 Magenta vessel system: (0.86+1.76)×{(0.96+1.0)/2}=2.6×

System Efficiency of electric knife in Oasis® media and the “smart” vessel system: (0.7+0.76)×{(0.96+1.1)/2}=1.4×

The systems efficiency was compared with a single cut by the scalpel and forceps from the agar and GA7 Magenta vessel system 1x. The stool cutting of two cycles increased the systems efficiency comparing with the single cut per culture. The electric knife was faster than the scalpel and forceps cutting tools but the regrowth of new T2 shoot tips was lower in Oasis® media than agar media which reduced the efficiency of the electric knife multiple-cut system.

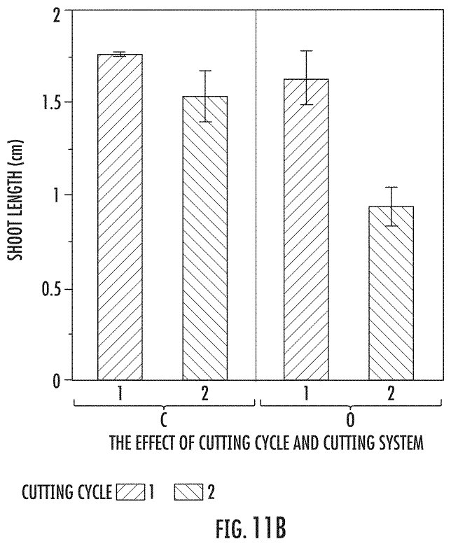

Plant quality in the greenhouse: The quality of the microcutting was measured by the growth in the greenhouse. Petunia cuts had 97±7% survival percentage after 2 weeks in the greenhouse. Media fed-batch techniques increased % rooted. LSMean differences student's t test analysis showed that the nutrient media fed-batch and batch culture were significantly difference from water fed-batch at 95% confidence. The % rooted from media fed-batch treatment was 90±11%, % rooted from batch culture was 87±11%, and % rooted from water fed-batch was 62±11%. Ragtime had longer shoots than Suncatcher (FIGS. 11A-11B). Plants had more leaves in T1 from scalpel and forceps in agar and GA7 Magenta vessel system than the electric knife multiple-cut system. Both Petunia genotypes had similar laboratory responses but the shoot length of Ragtime was longer than Suncatcher shoot in the greenhouse. Ragtime was chosen to run the next experiments to investigate the effect of media types at long-term culture.

### Example 2: Six Cycles of Multiple Cutting on Agar and Oasis® Using Scalpel and Forceps and Electric Knife for Ragtime

The experiment was performed to investigate the effect of long duration (three months), cutting cycles (six cycles), and fed-batch techniques on Petunia Ragtime shoot culture in the lab and the quality in the greenhouse.

### Materials and Methods

Plant material: Petunia x hybrida (3832 Ragtime) as described above.

In vitro micropropagation: Sixteen shoot tips were cultured in Oasis® media in the “smart” vessel (FIGS. 2 and 3A-3B) grown on 160 mL MS medium as described above. Six shoot tips grown on agargel MS medium in GA7 Magenta vessel as control system.

Cutting cycles and fed-batch techniques: The shoot tips were cut every two weeks with two cutting system: the electric knife in Oasis® media and the “smart” vessel (FIG. 2, FIGS. 3A-3B, and FIG. 5) and the scalpel and forceps in agar and GA7 Magenta vessel (FIG. 1). Cutting cycles were extended to six cycles through three months without subculture. The different fed-batch techniques were applied as described above.

Greenhouse acclimatization: Non-rooted shoot tips T1, T2, T3, T4, T5, and T6 were transferred directly to the soilless mix (Fafard 3B) for 2 weeks as described above.

Measurements: Multiplication ratio of T1, T2, T3, T4, T5, and T6 and plant cutting rate per min were counted in the laboratory. Survival %, % rooted, shoots length (cm), and numbers of leaves were counted after two weeks of growth in the greenhouse. The system efficiency was calculated. The calculations were as described above. The vessel initial mass and the vessel mass before and after the cutting were measured for each cycle and the water loss from the vessel was measured by:

Shoot tips fresh mass (g)=Vessel mass before cutting (g)−Vessel mass after cutting (g)

Water loss (mL)=Vessel initial mass (g)−Vessel mass before cutting (g)−Shoot tips fresh mass (g)

Experimental design and data analysis: A complete random design (2×3×6 full factorial) was conducted with two replicates of treatments and six replicates of the control. The factorial model terms were considered significant at P<0.05 (Table 4). The experiment design, analysis, and graphs were created using JMP version 12.0 (SAS Inst., Cary, N.C., USA).

### Results

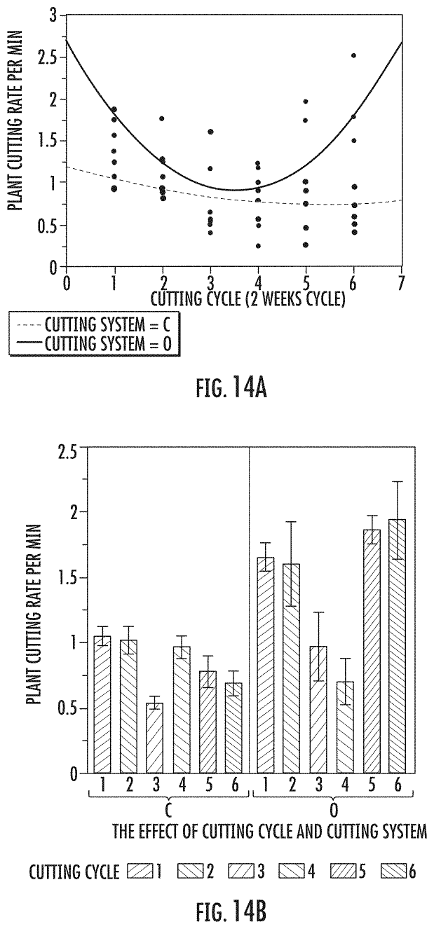

Labor components in the laminar flow hood: The multiplication ratio and plant cutting rate per min for the stool shoot cutting of six cycles were listed in Table 1. The scalpel and forceps in agar media and GA7 Magenta vessel had higher multiplication ratio in the first four cycles (T1 to T4) than the electric knife multiple-cut in Oasis® media and the “smart” vessel system (FIGS. 12A-12B). Shoot tips grown on Oasis® media had small root structure compared with agar media and that might clarify the low ratio of multiplication in T1 and T2 compared with the scalpel and forceps in agar media and GA7 Magenta vessel. After 4 weeks of growth T3 and T4 still had low multiplication ratio in Oasis® media compared with the agar media and GA7 Magenta vessel system, that could be due to the damage of the new small tips (next cut production) during the electric cutting (FIG. 13). The results showed that the regrowth of the new shoot tips reduced with the long cutting cycles in the GA7 Magenta vessel and that due to the limitation of nutrition and water in the small vessel. The multiplication ratio in the large “smart” vessel had a slight increase in T5 and T6 where the new shoot tips grow from the buds under the cutting level (FIGS. 12A-12B). The scalpel and forceps used 1.6±8.1 min/vessel to cut 6 shoot tips in GA7 Magenta vessel but the electric knife spent 0.73±8.1 min/vessel to cut 16 shoot tips in the “smart” vessel. The electric knife multiple-cut in Oasis® media and the “smart” vessel system had higher plant cutting rate per min than the scalpel and forceps in agar media and GA7 Magenta vessel system (FIGS. 14A-14B). The cutting time of the electric knife multiple-cut system in T3 and T4 cycles was longer (0.710.2 min/vessel) than the time used at T5 and T6 cycles (0.4±0.2 min/vessel). This increase in the cutting time due to the electric knife was brought back and forth through the vessel to cut the bending shoot tips. A grid may be added on the media surface to support shoot tissue during the cutting (see, e.g., FIGS. 7A-7C and FIGS. 24A-24B).

System Efficiency: After six cutting cycles, the total efficiency of the scalpel and forceps in agar and GA7 Magenta vessel system and electric knife in Oasis® and the “smart” vessel system were calculated using the numbers from Table 1 by the equations below:

System Efficiency of scalpel and forceps in agar and GA7 Magenta vessel system: (1.13+1.87+1.0+1.33+0.75+0.63)×{(1.05+1.0+0.53+0.96+0.77+0.68)/6}=8.39×

System Efficiency of electric knife multiple-cut in Oasis® media and the “smart” vessel system: (0.65+0.5+0.54+0.53+0.78+0.68)×{(1.6+1.6+0.96+0.69+1.85+1.93)/6}=7.95×

The stool cutting of six cycles improved the total efficiency of the scalpel and forceps from the agar and GA7 Magenta vessel and the electric knife multiple-cut in Oasis® media and the “smart” vessel systems comparing with a single cut by scalpel and forceps from the agar and GA7 Magenta vessel system 1x. Both systems had similar system efficiency with the long stool cutting.

Plants quality in the greenhouse: The electric knife multiple-cut in Oasis® media and the “smart” vessel system had 100±10% survival in the greenhouse through the cutting cycles but the scalpel and forceps in agar and GA7 Magenta vessel system was affected by the cutting cycles and reduced from 100±10% in T1 to 53±10% in T6. All the microcuttings had rooted in the greenhouse. The shoot length and the number of leaves of Ragtime, after 2 weeks in the greenhouse were affected by the cutting cycle. The quality of the microcutting (shoot length and number of leaves) was reduced through the long-term culture.

The electric knife multiple-cut in Oasis® media and the “smart” vessel system was faster than the scalpel and forceps in agar and GA7 Magenta vessel system but the damage of the small new shoot tips during the cutting might reduce the number of the shoot tips and that reduced the multiplication ratio. System efficiency of both cutting systems was the same but the plant from the electric knife in Oasis® media and the “smart” vessel system had greater survival in the greenhouse. Adding media or water to the Oasis® system may not provide an advantage on the system efficiency or the quality in the greenhouse. But the reduction of total volume from the evaporation and removing plants was higher in the batch treatment that lost 54±1.2 mL during the six cycles (12 weeks). The fed-batch techniques, media and water lost 2.0±2.2 mL and 0.54±1.6 mL respectively after 12 weeks. The reduction in the media volume was higher in the batch culture than fed-batch culture in the “smart” vessel. The results indicated that the water evaporation from the “smart” vessel not removing plant tissues (cutting) caused the loss in the volume of the batch culture although the ventilation membranes were closed with Aluminum foil to reduce the evaporation (FIG. 9B). The medium volume in Oasis® and the “smart” vessel (160 mL) may be adjusted.

### Example 3: 1-Four Cycles of Multiple Cutting on Agar and Oasis® Using Scalpel and Forceps and Electric Knife for Ragtime (2-Oasis® Media Volume I Experiment)

Two experiments were performed, one to compare the scalpel and forceps in agar and GA7 Magenta vessel system (“C”) with the electric knife multiple-cut in Oasis® and the “smart” vessel system (“0”) and in agar media and the “smart” vessel (“A”). The other experiment was performed to optimize the media volume in Oasis® media.

### Material and Methods

Plant material: Petunia x hybrida (3832 Ragtime) as described above.

In vitro micropropagation: Eight Petunia Ragtime shoot tips were grown on Oasis® media (160 mL, 200 mL, 240 mL, and 280 mL) in liquid MS medium as described above. Eight shoot tips grown on agargel media of 300 mL MS medium (300 mL medium volume was added to reach the height of Oasis® foam in the “smart” vessel so the cutting would be in the same level) supplemented with 0.65% w/v agargel (Sigma A3301) as described above. Six shoot tips grown on agargel MS medium in GA7 Magenta vessel as control system.

Cutting cycle systems: The shoot tips in the “smart” vessels were cut every three weeks with electric knife (FIG. 5). The shoot tips in GA7 Magenta vessels were cut with scalpel and forceps (FIG. 1) while recording the cutting time.

Measurements: Multiplication ratio of T1, T2, T3, and T4, plant cutting rate per min, and the length of the first three nodes in Petunia shoot (cm) were measured in a lab. The system efficiency and volume loss (mL) were calculated as described above.

Experimental design and data analysis: Two complete random designs (3×4 full factorial) and (Oasis® media volume I, 4×4) full factorial were conducted with four replicates of treatments and the control. The factorial model terms were considered significant at P<0.05 (Table 5 and Table 6, respectively). The experiment designs, analysis, and graphs were created using JMP version 12.0 (SAS Inst., Cary, N.C., USA).

### Results

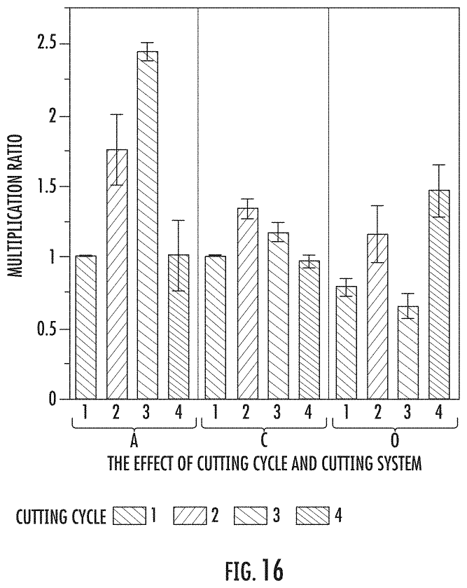

Labor components in the laminar flow hood: In the scalpel and forceps in agar and GA7 Magenta vessel system (“C”), multiplication ratio for the four cycles was 1.110.5 fold T1, 1.3±0.5 fold T2, 1.2±0.5 fold T3, and 0.95±0.5 fold T4 (FIG. 16). In the electric knife in Oasis® media and the “smart” vessel system (“0”) multiplication ratio for the four cycles was 0.78±0.5 fold T1, 1.15±0.5 fold T2, 0.65±0.5 fold T3, and 1.5±0.5 fold T4. In agar media and “smart” vessel (“A”) multiplication ratio for the four cycles was 1.0±0.5 fold T1, 1.8±0.5 fold T2, 2.4±0.5 fold T3, and 1.0±0.5 fold T4 (FIG. 16). The multiplication ratio of agar media and “smart” vessel system (“A”) was higher than the scalpel and forceps in agar and GA7 Magenta vessel system (“C”) where the plants grown in a large space in the “smart” vessel, W×L×H (100 mm×100 mm×127 mm dimensions) and get more medium (300 mL) comparing with 77 mm×77 mm×97 mm dimensions and medium volume of 50 mL in GA7 Magenta vessel. The electric knife in agar media and the “smart” vessel system (“A”) had the highest multiplication ratio at T2 and T3. In Oasis® media and the “smart” vessel system (“0”), the number of new shoot tips increased after the first cut at T2 then decreased at T3 and returned back to increase at T4. The cut by the electric knife damaged the small new tips that had short node length and that affected T3 multiplication ratio. The shoot tips T3 had a longer shoot node than T2 and that prevented the new tips near the buds from the damage during the cut and after three weeks T4 maximized to 1.510.5 fold. The multiplication ratio in Oasis® media improved with the long period cutting cycle (3 weeks).

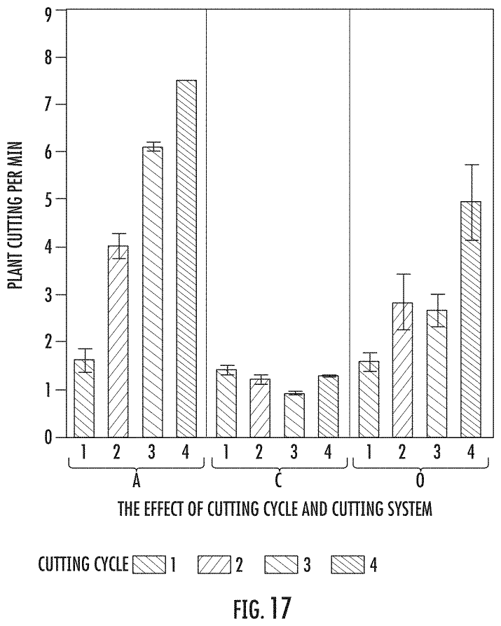

The scalpel and forceps used 1.0±0.1 min/vessel to cut 6 shoot tips in GA7 Magenta vessel but the electric knife used 0.41±0.1 min/vessel to cut 8 shoot tips from the “smart” vessel in both media “O” and “A”. Plant cutting rate per min for the scalpel and forceps in agar and GA7 Magenta vessel system was 1.4±1.2 min⁻¹ for T1, 1.21±1.2 min⁻¹ for T2, 0.9±1.2 min⁻¹ for T3, and 1.3±1.2 min⁻¹ for T4 (FIG. 17). The electric knife in Oasis® and the “smart” vessel system (“O”) had cutting rate of 1.6±1.2 min⁻¹ for T1, 2.8±1.2 min⁻¹ for T2, 2.6±1.2 min⁻¹ for T3, 4.9±1.2 min⁻¹ for T4. In agar media and “smart” vessel (“A”) had cutting rate of 1.6±1.2 min⁻¹ for T1, 4.0±1.2 min⁻¹ for T2, 6.1±1.2 min⁻¹ for T3, 7.5±1.2 min⁻¹ for T4. At the first cycle, the multiple-cut systems had similar plant cutting rate as the scalpel and forceps in agar and GA7 Magenta vessel system where the cutting was one plant per initial plant. The electric knife in agar media and the “smart” vessel (“A”) had the highest plant cutting rate in the later cycles, T2 to T4 and that reflected the speed of the electric knife and the regrowth of shoot tips from this system which is expressed as system efficiency. Media volume in Oasis® media had no significant effect on the multiplication ratio and plant cutting rate.

System Efficiency: After four cutting cycles, the total efficiency of the scalpel and forceps in agar and GA7 Magenta vessel system and electric knife in Oasis® media and in agar with the “smart” vessel systems were calculated using the numbers from the previous paragraphs by the equations below:

System Efficiency of scalpel and forceps in agar and GA7 Magenta vessel system: (1.1+1.3+1.2+0.95)×{(1.4+1.2+0.9+1.3)/4}=5.46×

System Efficiency of electric knife multiple-cut in Oasis® media and the “smart” vessel system: (0.78+1.2+0.65+1.5)×{(1.6+2.8+2.6+4.9)/4}=14.54×

System Efficiency of electric knife multiple-cut in agar media and the “smart” vessel system: (1.3+1.8+2.4+1.0)×{(1.6+4.0+6.1+7.5)/4}=31.2×

Agar media in the “smart” vessel had the highest system efficiency comparing with a single cut by the scalpel and forceps from the agar and GA7 Magenta vessel system 1x and the other systems.

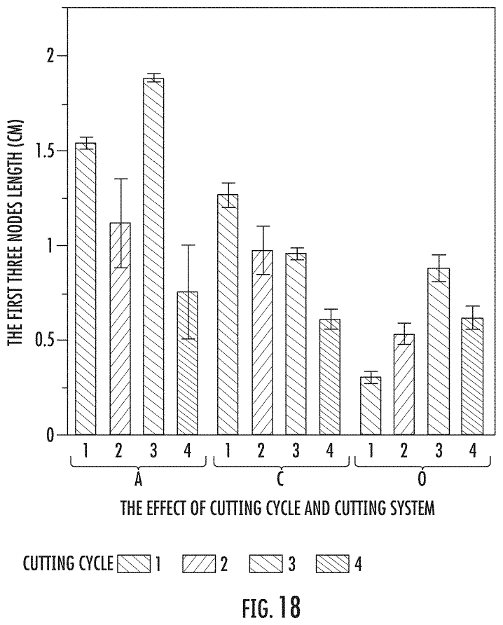

Plant quality in laboratory: Shoot node length was measured after the cut directly to test the quality of the stool shoots in different cutting systems. The scalpel and forceps in agar and GA7 Magenta vessel system (“C”) had shoot node 1.1±0.2 cm from T1 cycle and reduced to 0.5±0.2 cm at T4 cycle (FIG. 18). The electric knife in Oasis® media and the “smart” vessel system (“0”) had the lowest shoot node at the T1 cycle (0.3±0.2 cm) and increased to 0.7±0.2 cm at T4 cycle. However, multiple-cut in agar media and the “smart” vessel (“A”) had the highest shoot node length (1.5±0.2 cm) from T1 cycle and reduced through cutting cycles to 0.96±0.2 cm at T4 cycle (FIG. 18). Petunia Ragtime had greater microcuttings and high quality in the multiple-cut in agar and the “smart” vessel system “A” than “0” and “C” systems. The length of shoot node might affect the efficiency of the cutting system, where the electric knife could damage the new regrowth tips during the cut and that could reduce the multiplication ratio for the next cutting cycle.

### Example 4: 1-Four Cycles of Multiple Cutting on Agar and Oasis® Using Scalpel and Forceps and Scissor for Ragtime (2-Oasis® Media Volume II Experiment)

Two experiments were performed, one to compare the scalpel and forceps in agar and GA7 Magenta vessel system (“C”) with the scalpel and forceps in Oasis® media and the “smart” vessel system (“KO”) and in agar media and the “smart” vessel (“KA”). Scissors were tested as a cutting tool in Oasis® media and the “smart” vessel (“SO”) and in agar media and the “smart” vessel (“SA”). The other experiment was performed to optimize the media volume in Oasis® media.

### Material and Methods

Plant material: Petunia x hybrida (3832 Ragtime) as described above.

In vitro micropropagation: Eight Petunia Ragtime shoot tips were grown on Oasis® media (160 mL, 200 mL, 240 mL, and 280 mL) in MS medium as described above. Eight shoot tips grown on agargel media of 280 mL MS medium supplemented with 0.65% w/v agargel (Sigma A3301) as described above. Six shoot tips grown on agargel MS medium in GA7 Magenta vessel as control system.

Cutting cycle systems: The shoot tips in the “smart” vessels were cut every two weeks with scissors (“SA” and “SO”) (see, e.g., FIG. 6) and scalpel and forceps (“KA” and “KO”) (see, e.g., FIG. 1). The shoot tips in GA7 Magenta box were cut with scalpel and forceps as a control system (“C”) while recording the cutting time.

Measurements: Multiplication ratio of T1, T2, T3, and T4, plant cutting rate per min and the length of the first three nodes in Petunia shoot (cm), shoot tips fresh mass (g) were counted in a lab. System efficiency was calculated for each cutting system as described above.

Experimental design and data analysis: Two complete random designs (5×4 full factorial) and (Oasis® media volume II, 4×4) full factorial were conducted with four replicates of treatments and the control. The factorial model terms were considered significant at P<0.05 (Table 7 and Table 8, respectively). The experiment designs, analysis, and graphs were created using JMP version 12.0 (SAS Inst., Cary, N.C., USA).

### Results

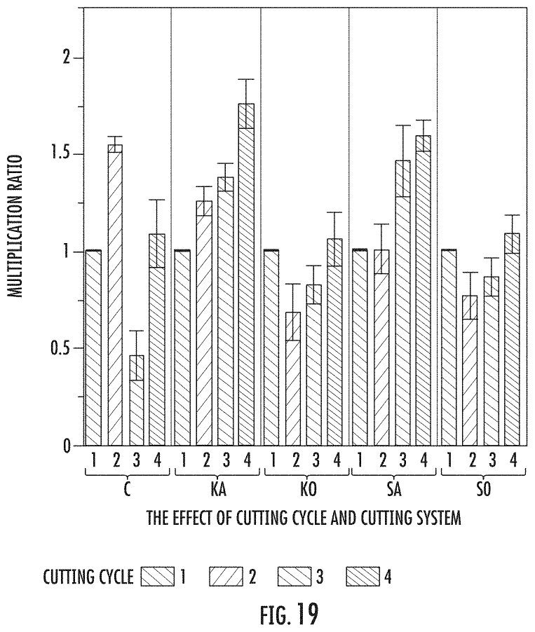

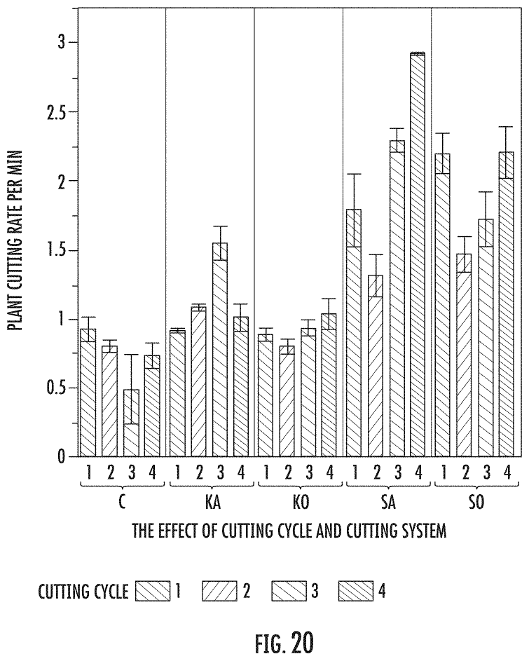

Labor components in the laminar flow hood: The multiplication ratio and cutting rate per min for the four cycles in all cutting systems are list in Table 2. All systems had 1.0±0.2 fold multiplication ratio at T1 cycle (FIG. 19). The continuous cutting by using the scalpel and forceps in agar media and GA7 Magenta vessel (“C”) increased the shoot tips regrowth (T2) however, the flowering tips increased at T3 and T4 cycles. Cutting tools, the scalpel and forceps and scissors, in agar media and the “smart” vessel (“KA” and “SA”) increased the multiplication ratio through the cutting cycles (FIG. 19) and had the highest multiplication ratio at T4 (1.8±0.2 and 1.6±0.2 fold). Oasis® media and the “smart” vessel with both cutting tools reduced the multiplication ratio at T2 then the regrowth of the shoot tips increased up to 1.0±0.2 fold. Oasis® media had lower multiplication ratio than agar in the “smart” vessel (Table 2); root structure in agar media was bigger than in Oasis® media as observed from first cycle through the forth cycles. Increase Oasis® media volume up to 240 mL increased the multiplication ratio after the stool shoot.

However, scissors in agar media and the “smart” vessel increased the cutting rate twice (2.1±0.1 min⁻¹) as the scalpel and forceps in agar media and GA7 Magenta vessel (1.0±0.1 min⁻¹) Scissors had higher plant cutting rate (1.9±0.1 min⁻¹) than scalpel and forceps (0.9±0.1 min⁴) in Oasis® media and the “smart” vessel. Scissors in Oasis® and agar media and the “smart” vessel (“SA” and “SO”) had its highest plant cutting rate per min than other systems (“C”, “KA”, and “KO”) (FIG. 20).

System Efficiency: The systems efficiency was compared with a single cut by scalpel and forceps from the agar and GA7 Magenta vessel system 1x. After four cutting cycles, the total efficiency of the scalpel and forceps in agar and GA7 Magenta vessel system (“C”) and scalpel and forceps in Oasis® media and agar with the “smart” vessel systems (“KO” and “KA”), and scissors in Oasis® media and agar with the “smart” vessel systems (“SO” and “SA”) were calculated using the numbers from Table 2 by the equations below:

System Efficiency of scalpel and forceps in agar and GA7 Magenta vessel system (“C”): (1.0+1.5+0.46+1.0)×{(0.92+0.80+0.48+0.73)/4}=2.9×

System Efficiency of scalpel and forceps in Oasis® media and the “smart” vessel system (“KO”): (1.0+0.68+0.82+1.0)×{(0.88+0.79+0.92+1.0)/4}=3.14×

System Efficiency of scalpel and forceps in agar media and the “smart” vessel system (“KA”): (1.0+1.3+1.4+1.75)×{(0.91+1.1+1.5+1.0)/4}=8.7×

System Efficiency of scissor in Oasis® media and the “smart” vessel system (“SO”): (1.0+0.76+0.86+1.0)×{(2.2+1.5+1.7+2.2)/4}=10.3×

System Efficiency of scissor in agar media and the “smart” vessel system (“SA”): (1.0+1.0+1.5+1.6)×{(1.8+1.3+2.3+2.9)/4}=10.6×

The system efficiency of scissor in agar or Oasis® media and the “smart” vessel was higher than the scalpel and forceps in GA7 Magenta and the “smart” vessels. The efficiency of scalpel and forceps in agar media was higher than Oasis® media in the “smart” vessel.

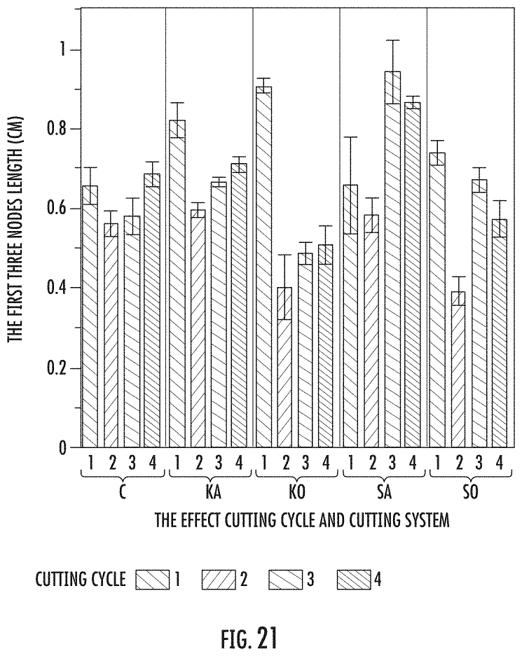

Plant quality in the laboratory: Shoot node length and fresh mass were measured after the cut directly to test the quality of the stool shoots in different cutting systems. The scalpel and forceps in agar and GA7 Magenta vessel system had 0.6±0.1 cm lengths of the three first nodes (tips cut) through cutting cycles (FIG. 21). The length of shoot nods was higher at first cut T1 at the “smart” vessel systems then the length decreased at T2; after that the length of the new shoot tips increased at T3 and T4 (FIG. 21). The length of the first three nodes was longer from agar media and “smart” vessel than Oasis® media through all cycles and reached 0.9±0.1 cm at T3 and T4-SA.

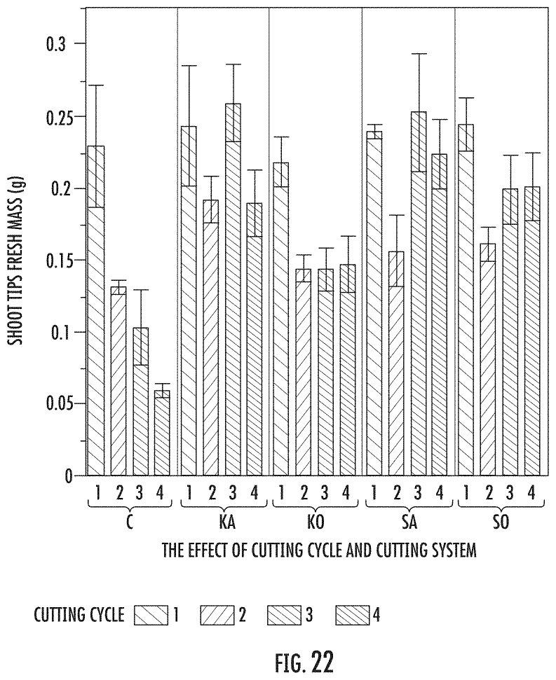

All cutting systems had shoot tips with 0.2±0.05 g fresh mass at T1 cycle then the fresh mass reduced after the first cut T1 (FIG. 22). The fresh mass of the shoot tips from the scalpel and forceps in agar and GA7 Magenta vessel system reduced from 0.23 to 0.06±0.05 g through the cutting cycle (FIG. 22). In agar media and the “smart” vessel systems (“KA” and “SA”), the fresh mass of shoot tips started to increase after the third cut T3 as well in scissors and Oasis® media and the “smart” vessel system (SO) and reached 0.2±0.05 g at T4 cycles. The scalpel and forceps in Oasis® media and the “smart” vessel system (“KO”) had no increase in shoot tips fresh mass through T2, T3, and T4. The high volume media (280 mL) in Oasis® had the highest fresh mass through all cutting cycles (0.2±0.05 g).

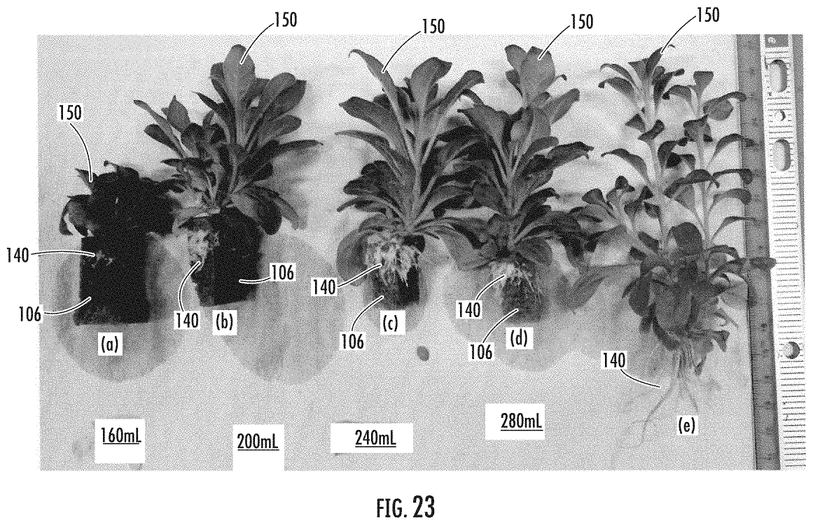

The quality of Ragtime microcutting was higher in agar media than Oasis® media and the “smart” vessel system. Shoot and root systems in agar media and the “smart” vessel was larger than plants from Oasis® media and the “smart” vessel (FIG. 23).

### Example 5: Six Cutting Cycles of Multiple-Cut System Using Blade and Grid for Fed-Batch Micropropagation of Petunia Ragtime Stool Shoots Rooted in Agar and Oasis® Foam

This experiment was performed to compare the scalpel and forceps in agar and GA7 Magenta vessel system (“C”) with the scalpel and forceps in Oasis® media and the “smart” vessel system (“KO”) and in agar media and the “smart” vessel (“KA”). A blade and grid were tested as cutting tools in Oasis® media and the “smart” vessel (“BO”) and in agar media and the “smart” vessel (“BA”). Fed-batch technique was tested only in Oasis® media and the “smart” vessel system.

### Material and Methods

Plant material: Petunia x hybrida (3832 Ragtime) as described above.

Six shoot tips were grown in a GA7 Magenta vessel (Magenta Corp., Chicago, Ill., USA) in a cooler room 12° C. for 42 weeks under 20 μmol m⁻²s⁻¹ light intensity (GreenPower LED production module deep red/blue 120 II0V, Philips, USA). Following the storage period, shoot tips were cut and six shoot tips (T0) were cultured on 50 mL MS medium described above in the Magenta vessel at 23±2° C. and under 30 μmol m⁻²s⁻¹ cool white fluorescent lights for 16 hours a day. After two weeks, T1 microcuttings from the six T0 plants were cut using scalpel and forceps. Sixteen shoot tips T1 were cultured in the “smart” vessel on 280 mL MS medium described above. After two weeks T2 shoot tips were used in the experiment. Each subsequent cycle of tipping is designated Tn, where n=number of times tip removal was performed.

In vitro micropropagation: In the “smart” vessel (FIG. 2 and FIGS. 3A-3B), a large size vessel, (W×L×H (100 mm×100 mm×127 mm dimensions), sixteen shoot tips (4×4 rows) grown on 280 mL agar MS medium supplemented with 0.65% w/v agargel (Sigma A3301) as described above. Sixteen shoot tips were cultured on Oasis® media (Grower Solutions, Kent, Ohio, USA), on 280 mL in liquid MS medium as described above without the agargel. Six shoot tips (3×2 rows) grown on agargel MS medium in GA7 Magenta vessel (77 mm×77 mm×97 mm dimensions) as a control system (“C”).

Cutting cycle systems: The scalpel and forceps cut T1 microcuttings after two weeks. A sterile steel grid was put on the top of the plants (FIGS. 24A-24B) in the half of the “smart” vessels per treatment to support the shoots cutting by the blade. The shoot tips in the “smart” vessels were cut every two weeks with the blade and grid (FIG. 25) and the scalpel and forceps. The shoot tips in the GA7 Magenta vessel were cut with the scalpel and forceps while recording the cutting time (min). At the time of the cutting, the fed-batch technique was applied to reset the volume of the media to the initial volume (280 mL) as in the following equation:

The media to add mL=Initial vessel mass with the plant tissue and media+grid mass (if it is there)−the vessel mass after the cutting

Cutting schedule: Experiment started on August 30^{th}
T1: September 13^{th}
T2: September 27^{th}
T3: October 11^{th}
T4: October 25^{th}
T5: November 8^{th}
T6: November 21^{th}

Measurements: Multiplication ratio of T1, T2, T3, T4, T5, and T6, cutting time (min), plant cutting rate per min, the length of the average of first three nodes in Petunia shoot (cm), the average of fresh mass of each microcutting (g), flowering tips %, hyperhydricity %, and water lost per vessel were measured in the laboratory. Shoot node length of the first three leaves was measured directly after the cut.

Multiplication ratio=(the number microcuttings−hyperhydricity tips)/initial shoot tips in the vessel

Plant cutting rate per min=the multiplication ratio/the cutting time (min)

The system efficiency was calculated by using the equation below:

System efficiency=the sum of multiplication ratio for each cutting cycle×the average of plant cutting rate of the cutting cycles

Water lost=the vessel initial mass (g)−the vessel mass before the cutting (g)

Experimental design and data analysis: The complete random design of cutting type, media type, cutting cycles, and fed-batch technique (2×2×2×5 full factorial) were conducted with two replicates of each treatment and four replicates of the control. The full factorial models with quadratic term of cutting cycle were selected by stepwise forward method and model terms were considered significant at P<0.05. The effect of factors on the responses, cutting time and cutting rate per min was tested by t-student test at 95% confidence interval. Design, data analysis, and graphs were created using JMP version 12.0 (SAS Inst., Cary, N.C., USA).

### Results

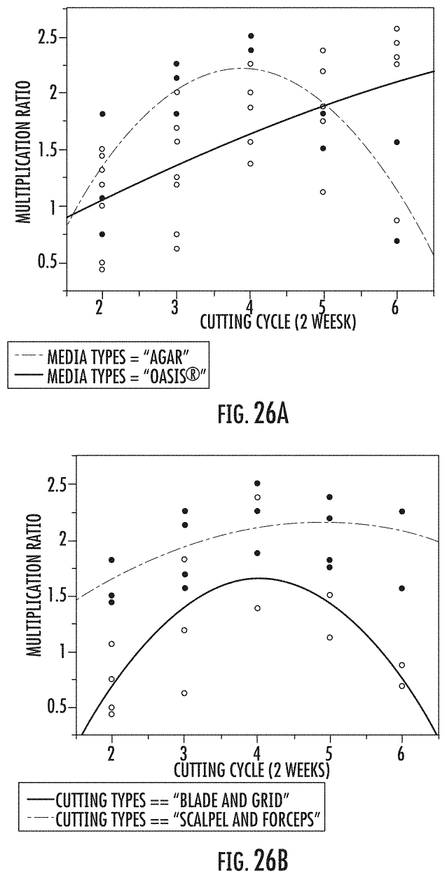

Labor components in the laminar flow hood: The multiplication ratio was affected significantly by the quadratic term of cutting cycles, the main terms of cutting type, media type and their interactions with cutting cycle. In the scalpel and forceps cutting system, the multiplication ratio reached the maximum (2.2±0.3 fold) in T4 in agar medium and in T5 in Oasis® medium (FIGS. 26A-26B). In the sixth cycle, the multiplication ratio was 2.1±0.3 fold in Oasis® media and 1.7±0.3 fold in the agar media. In the blade and grid cutting system, the multiplication ratio reached the maximum at T4 (1.7±0.3 fold) in agar medium and (1.2±0.3 fold) at T4 and T5 in Oasis® media. The scalpel and forceps cutting system had higher multiplication ratio than the blade and grid cutting system in both media types. The fed-batch treatments may have no significant effect on Petunia multiplication ratio. In GA7 Magenta vessel and the scalpel and forceps cutting system, the multiplication ratio in T2 was 1.7±0.5 fold and reduced with cutting cycle to 0.72±0.5 fold in T6.

The cutting type, the cutting cycles and their interaction could significantly affect the cutting time and plants cutting rate. In the smart vessel, the scalpel and forceps cutting system had longer cutting time (5.3±0.3 min/vessel) than the blade and grid cutting system (0.46±0.3 min/vessel). Thus, the blade and grid cutting system was 11 times faster than the scalpel and forceps cutting system. The plant cutting rate using the blade and grid was 2.7±0.6 min⁻¹ in T2 and reduced to 4.13±0.6 min⁻¹ in T6 in the agar media and 2.0±0.6 min⁻¹ in T2 and reduced to 4.77±0.6 min⁻¹ in T6 in Oasis® media. In the scalpel and forceps cutting system, plant cutting rate was 0.6±0.6 min⁻¹ in T2 and reduced to 0.29±0.6 min⁻¹ in T6 in the agar media and 0.4±0.6 min⁻¹ in T2 and reduced to 0.36±0.6 min⁻¹ in T6 in Oasis® media. The media type and fed-batch had no effects on the cutting time. The blade and grid system was faster than the scalpel and forceps cutting system and the blade and grid system could produce more microcuttings per min. In the GA7 magenta vessel with the scalpel and forceps cutting system, the cutting rate had 0.98±0.3 min⁻¹ in T2 then reduced to 0.83±0.3 min⁻¹ in T6.

System Efficiency: After six cutting cycles, the total efficiency of the different cutting systems were calculated by using the equation:

System efficiency=the sum of multiplication ratio for each cutting cycle×the average of plant cutting rate of the cutting cycles

The first cutting cycles was not considered because the blade and grid were applied after the first cut. The efficiency was calculated for the last five cycles.

System efficiency of the scalpel and forceps in the agar and the “smart” vessel system (“KA”): (1.9+2.2+2.2+2.1+1.7)×{(0.60+0.33+0.54+0.45+0.29)/5}=4.5×

System efficiency of blade and grid in agar and the “smart” vessel system (“BA”): (1.3+1.6+1.7+1.5+1.2)×{(2.7+8.78+11.05+5.0+4.13)/5}=46.2×

System efficiency of the scalpel and forceps in Oasis© and the “smart” vessel system (“KO”): (1.2+1.8+2.1+2.2+2.1)×{(0.42+0.29+0.39+0.27+0.36)/5}=3.3×

System efficiency of the blade and grid in Oasis® and the “smart” vessel system (“BO”): (0.5+0.96+1.2+1.2+0.97)×{(2.02+7.34+8.02+4.82+4.77)/5}=26.0×

System efficiency of the scalpel and forceps in GA7 Magenta vessel system (“C”): (1.7+1.8+1.7+1.9+1.3+0.72)×{(0.98+1.02+0.95+0.87+0.83)/5}=8.5×

The systems efficiency was compared with a single cut by the scalpel and forceps from the agar and GA7 Magenta vessel system 1x. The stool cutting of six cycles improved the total efficiency of the scalpel and forceps from the agar and Oasis® media in the “smart” vessel system and in agar GA7 Magenta vessel and the blade and grid in agar and Oasis® media and the “smart” vessel systems compering with a single cut by scalpel and forceps from the agar and GA7 Magenta vessel system 1x. In the “smart” vessel, both multiple cutting systems in agar medium had higher system efficiency than the multiple cutting systems in Oasis® medium. The blade and grid cutting system had the highest efficiency than the scalpel and forceps systems. In the scalpel and forceps cutting system, the efficiency was higher in GA7 Magenta vessel system than in the efficiency of the “smart” vessel systems.

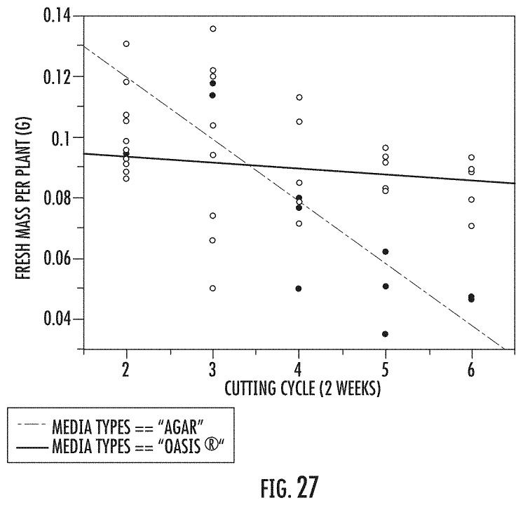

Plant quality in laboratory: The cutting cycle and its interaction with media types had significant effects on the fresh mass of Petunia shoot tips (FIG. 27). The fresh mass from the “smart” vessel system and agar medium was higher than Oasis® medium in T2 (0.12±0.01 g; 0.09±0.01 g respectively) but the fresh mass in agar medium reduced with the cutting cycle to 0.08±0.01 g in T4 and to 0.04±0.01 g in T6 in agar medium. Plants in Oasis® medium had constant fresh mass (0.09±0.01 g) without any significant effect to the fed-batch treatments. Cutting type had no significant effect on the fresh mass of shoot cuts. In the GA7 magenta vessel and the scalpel and forceps cutting system, the fresh mass of cuts was 0.07±0.02 g in T2 and reduced to 0.04±0.02 g in T6. In agar media, the fresh mass reduced with the cutting cycles compared to the liquid media in Oasis® that had a constant fresh mass through the cycles (12 weeks).

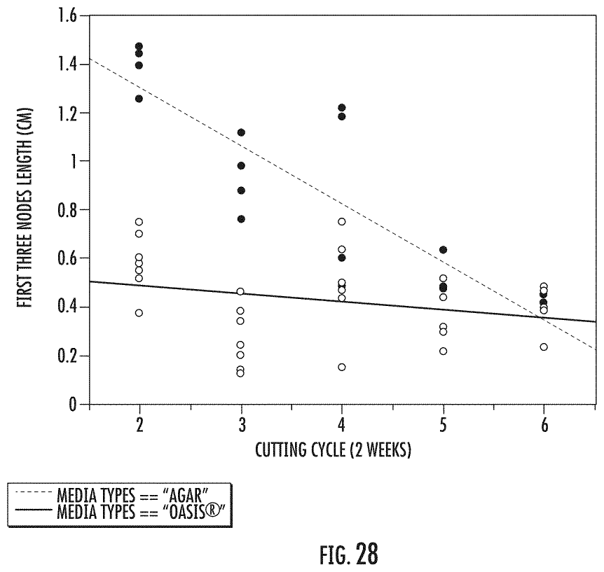

The media types, cutting cycle and their interaction significantly affected the shoot nodes length (FIG. 28). In the agar medium, shoot nodes length was 1.39±0.2 cm in T2 and reduced to 0.46±0.2 cm in T6. And in Oasis® media, shoot nodes length was 0.57±0.2 cm in T2 and reduced to 0.38±0.2 cm in T4, and T5 but it elongated back in T6 to 0.43±0.2 cm. The shoot nodes length from GA7 magenta vessel and the scalpel and forceps cutting system reduced from 0.9±0.2 cm in T2 to 0.3±0.2 cm in T6. Petunia shoot cut's quality in agar declined faster with the cutting cycles but plants from Oasis® medium were more stable through the cutting cycles.

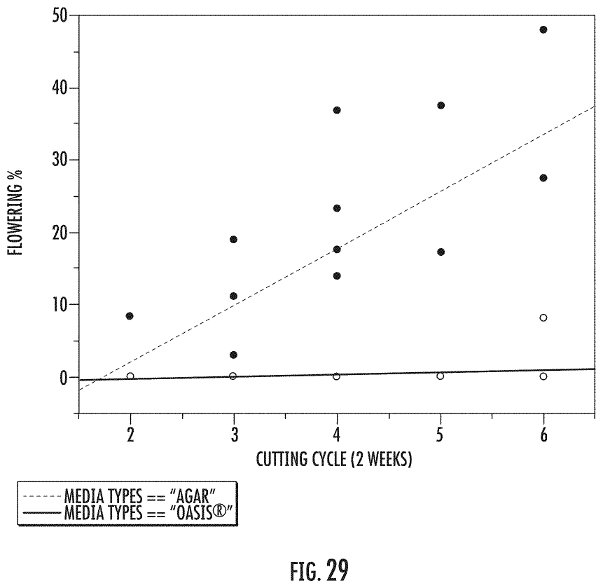

Flowering %: The flowering % was significantly affected by media types, cutting cycle and their interaction (FIG. 29). The flowering tips appeared early in T2 (5.016.6%) and increased through the cutting cycles to reach 37.1±6.6% in T6 in the agar media. Plants in Oasis® media had 0.916.6% flowering tips in T2 and 1.3±6.6% in T6. In the GA7 magenta vessel and the scalpel and forceps cutting system, plants in T2 had 15.7±21.3% of shoot tips reached the mature stage, flowering. In T6 cycle 61.5±21.3% of the shoot tips reached the mature stage. Plants grown on agar media reached mature stage faster than plants grown on Oasis® medium.

Hyperhydricity %: The media types, cutting cycle, cutting types and their interactions, and fed-batch significantly affected the hyperhydricity percentage per vessel (FIG. 30). The hyperhydric tips appeared only in Oasis® media and increased with the cycles. In the scalpel and forceps cutting system, the percentage of hyperhydricity of T3 was 7.7±7.5% with fed-batch treatment and 2.5±7.5% without fed-batch treatment. In the blade and grid cutting system, the hyperhydricity % of T3 was 13.9±7.5% with fed-batch treatment and 8.7±7.5% without fed-batch treatment. In the scalpel and forceps cutting system, the hyperhydricity % of T6 increased to 7.2±7.5% without fed-batch and to 14.8±7.5% with fed-batch treatment. In the blade and grid cutting system, the hyperhydricity % of T6 increased to 35.2±7.5% without fed-batch and to 60.8±7.5% with fed-batch treatment. The plants from the agar media in the “smart” or GA7 magenta vessels had no hyperhydricity shoots through the cutting cycles.

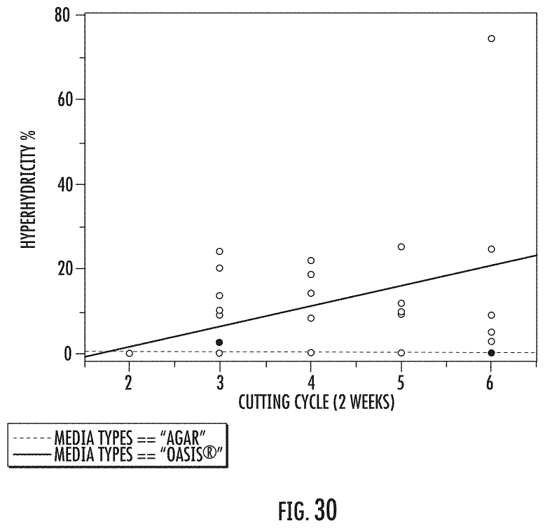

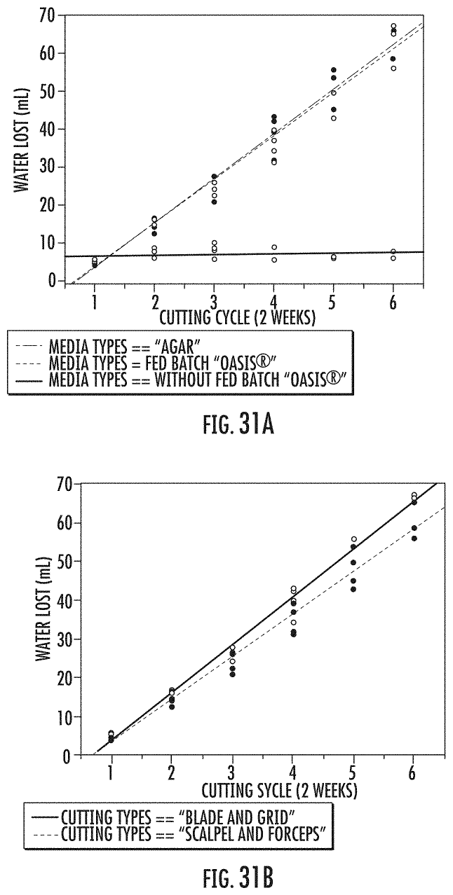

Water lost: Losing water from the “smart” vessel was affected by fed-batch treatments, cutting type, their interaction with cutting cycle, and cutting cycle. The vessels with the scalpel and forceps cutting system lost 7.0±2.5 mL of water after 12 weeks with fed-batch treatment and lost 58.1±2.5 mL of water after 12 weeks without fed-batch treatment (FIGS. 31A-31B). In the blade and grid cutting system, the water lost was 14.1±2.5 mL after 12 weeks with fed-batch treatment and lost 65.2±2.5 mL of water after 12 weeks without fed-batch treatment. The media type had no effect on the water loss from the vessel. The reduction in the media volume was higher in the batch culture than fed-batch culture in the “smart” vessel. The blade and grid system increased the loss of water from the vessel. The pressure of the grid on the medium might increase the evaporation rate of the water from the vessel by compressing the matrix and forcing liquid water to be expressed. The results indicated that the water evaporation from the “smart” vessel not removing plant tissues (cutting) caused the loss in the volume of the batch culture although the ventilation membranes were closed with aluminum foil to reduce the evaporation (FIG. 8B).

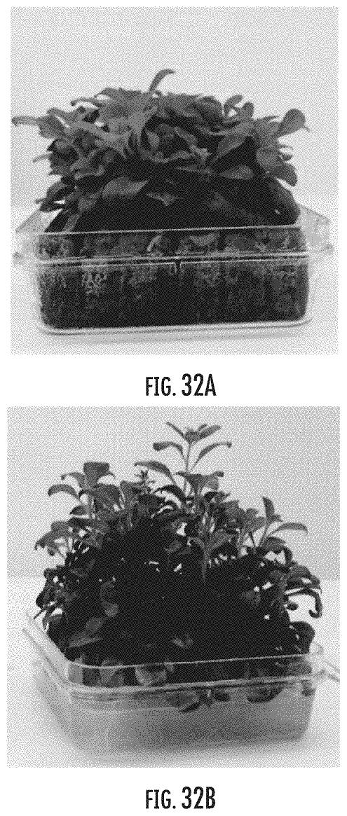

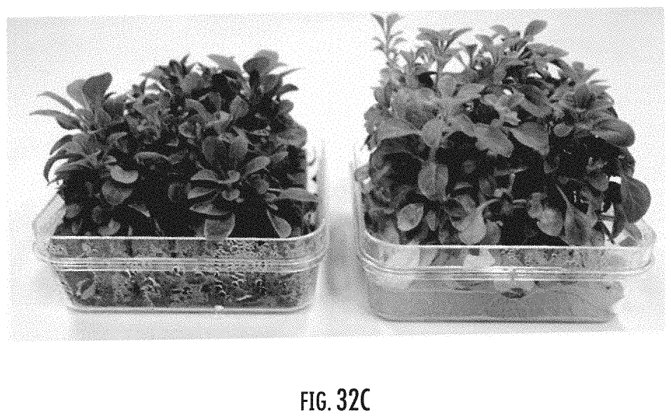

The “smart” vessel systems with the two different types of cuttings with agar produced more plants and higher quality than Oasis® media, but with the continuous cutting, the multiplication ratio, fresh mass and the length of the microcuttings were reduced on agar medium (FIGS. 32A-32C). Furthermore, plants in agar reached the flowering stage faster than plants in Oasis® media. However, Oasis®'s plants were more stable in the quality, fresh mass and shoot length and the production of microcuttings still reasonable with the scalpel and forceps cutting system. Plants from Oasis® medium showed an increase in the quality with the time with no need to add more medium to the system. Fed-batch treatment (adding media after the cutting) increased the hyperhydricity %.

In agar media, the “smart” vessel had more and larger shoot tips through the 12 weeks than GA7 Magenta vessel. The plant growth might be influenced by the large space of the “smart” vessel. The large vessels increased the fresh mass yield. That large space and the big number of plant reduce the system efficiency of scalpel and forceps cutting system comparing with GA7 Magenta vessel.

The grid had a negative effect on Petunia multiplication ratio and increased the hyperhydricity % and the water lost from the vessel. The grid might compress the Oasis® media. This pressure might affect the plant growth and increase buildup the water around the root in the Oasis® medium which increases the hyperhydricity % and water evaporation from the vessel. The volume of the medium may be reduced to control these problems the aluminum cover from the pores.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. The invention is defined by the following claims, with equivalents of the claims to be included therein.

TABLE 1
The multiplication ratio and plant cutting rate for Ragtime
in the six cutting cycles for scalpel and forceps in
agar and GA7 Magenta vessel system (C) and the electric
knife in Oasis ® and the “smart” vessel system (O)

| Cutting | Cutting | Multiplication | Plant cutting |
|---|---|---|---|
| system | cycles | ratio | rate per min |
| System C | T1 | 1.13 ± 0.2 | 1.05 ± 0.2 |
| System C | T2 | 1.87 ± 0.2 | 1.0 ± 0.2 |
| System C | T3 | 1.0 ± 0.2 | 0.53 ± 0.2 |
| System C | T4 | 1.33 ± 0.2 | 0.96 ± 0.2 |
| System C | T5 | 0.75 ± 0.2 | 0.77 ± 0.2 |
| System C | T6 | 0.63 ± 0.2 | 0.68 ± 0.2 |
| System O | T1 | 0.65 ± 0.2 | 1.6 ± 0.2 |
| System O | T2 | 0.5 ± 0.2 | 1.6 ± 0.2 |
| System O | T3 | 0.54 ± 0.2 | 0.96 ± 0.2 |
| System O | T4 | 0.53 ± 0.2 | 0.69 ± 0.2 |
| System O | T5 | 0.78 ± 0.2 | 1.85 ± 0.2 |
| System O | T6 | 0.68 ± 0.2 | 1.93 ± 0.2 |

TABLE 2
The multiplication ratio and plant cutting rate for Ragtime
in the four cutting cycles for scalpel and forceps in agar
and GA7 Magenta vessel system (C), the scalpel and forceps
(KO) and scissor (SO) in Oasis ® and the “smart”
vessel system, and the scalpel and forceps (KA) and scissor
(SA) in agar and the “smart” vessel system (A)

| Cutting | Cutting | Multiplication | Plant cutting |
|---|---|---|---|
| system | cycles | ratio | rate per min |
| System C | T1 | 1.0 ± 0.2 | 0.92 ± 0.2 |
| System C | T2 | 1.5 ± 0.2 | 0.8 ± 0.2 |
| System C | T3 | 0.46 ± 0.2 | 0.48 ± 0.2 |
| System C | T4 | 1.0 ± 0.2 | 0.73 ± 0.2 |
| System KO | T1 | 1.0 ± 0.2 | 0.88 ± 0.32 |
| System KO | T2 | 0.68 ± 0.2 | 0.79 ± 0.2 |
| System KO | T3 | 0.82 ± 0.2 | 0.92 ± 0.2 |
| System KO | T4 | 1.0 ± 0.2 | 1.0 ± 0.2 |
| System SO | T1 | 1.0 ± 0.2 | 2.2 ± 0.2 |
| System SO | T2 | 0.76 ± 0.2 | 1.5 ± 0.2 |
| System SO | T3 | 0.86 ± 0.2 | 1.7 ± 0.2 |
| System SO | T4 | 1.0 ± 0.2 | 2.2 ± 0.2 |
| System KA | T1 | 1.0 ± 0.2 | 0.91 ± 0.2 |
| System KA | T2 | 1.3 ± 0.2 | 1.1 ± 0.2 |
| System KA | T3 | 1.4 ± 0.2 | 1.5 ± 0.2 |
| System KA | T4 | 1.75 ± 0.2 | 1.0 ± 0.2 |
| System SA | T1 | 1.0 ± 0.2 | 1.8 ± 0.2 |
| System SA | T2 | 1.0 ± 0.2 | 1.3 ± 0.2 |
| System SA | T3 | 1.5 ± 0.2 | 2.3 ± 0.2 |
| System SA | T4 | 1.6 ± 0.2 | 2.9 ± 0.2 |

TABLE 3
Summary ANOVA of multiple cutting on agar and Oasis ® with scalpel
and forceps and electric knife using two genotypes of Petunia

| | Prob > F of responses | | | | | |
|---|---|---|---|---|---|---|
| | Laboratory | | Greenhouse | | | |
| Terms | Multiplication | Plant cutting | Surv. | % | Shoot | Leaves |
| Source of Variation | ratio | rate per min | % | Rooted | cm | number |
| Petunia | 0.3938 | 0.9919 | 0.4900 | 0.5473 | 0.0001* | 0.0959 |
| Cutting cycle | 0.0103* | 0.9081 | 0.7283 | 0.5760 | 0.0001* | 0.0286* |
| Fed-batch techniques | 0.3802 | 0.7822 | 0.3171 | 0.0178* | 0.7442 | 0.4299 |
| Cutting type | 0.0145* | 0.0112* | 0.1859 | 0.3484 | 0.0423* | 0.0403* |
| Petunia*Cutting type | 0.1575 | 0.6341 | 0.0966 | 0.6283 | 0.6132 | 0.5267 |
| Cutting cycle*Cutting type | 0.0348* | 0.8084 | 0.6848 | 0.9113 | 0.1470 | 0.2210 |
| Petunia*Cutting cycle*Cutting type | 0.0508 | 0.3275 | 0.4087 | 0.8566 | 0.6883 | 0.7292 |
| Petunia*Cutting cycle | 0.7675 | 0.2646 | 0.4696 | 0.2047 | 0.0486* | 0.3407 |
| Petunia*Fed-batch techniques | 0.2974 | 0.7597 | 0.3171 | 0.7796 | 0.3208 | 0.5146 |
| Cutting cycle*Fed-batch techniques | 0.5851 | — | 0.5620 | 0.9815 | 0.8211 | 0.8659 |
| Petunia*Cutting cycle*Fed-batch | 0.4510 | — | 0.5620 | 0.6297 | 0.0649 | 0.4601 |
| techniques | | | | | | |
| Whole model fit (R²) | 0.630*** | 0.392 | 0.416 | 0.444 | 0.784*** | 0.581* |

TABLE 4
Summary ANOVA from six cycles of multiple cutting on agar and Oasis ® using
scalpel and forceps and electric knife for Ragtime

| | Prob > F of responses | | | | |
|---|---|---|---|---|---|
| | Laboratory | | Greenhouse | | |
| Terms | Multiplication | Plant cutting | Survival | Shoot | Leaves |
| Source of Variation | ratio | rate per min | % | cm | number |
| Cutting cycle | 0.1316 | 0.5605 | 0.0312* | 0.0001* | 0.0001* |
| Cutting system | 0.0001* | 0.0410* | 0.6324 | 0.1794 | 0.0550 |
| Fed-batch techniques | 0.9464 | 0.6176 | 0.9145 | 0.5857 | 0.3858 |
| Cutting cycle* Cutting system | 0.0625 | 0.5574 | 0.0094* | 0.3961 | 0.8794 |
| Fed-batch techniques*Cutting cycle | 0.6507 | 0.9946 | 0.5471 | 0.7199 | 0.2433 |
| (Cutting cycle)² | 0.4707 | 0.0045* | 0.0343* | 0.0105* | 0.5151 |
| Whole model fit (R²) | 0.527*** | 0.426* | 0.284* | 0.657*** | 0.752*** |

TABLE 5
Summary ANOVA from four cycles of multiple cutting on
agar and Oasis ® using scalpel and forceps
and electric knife for Ragtime experiment

| | Laboratory Prob > F | | |
|---|---|---|---|
| | Multipli- | Plant | The first |
| Terms | cation | cutting rate | three nodes |
| Source of Variation | ratio | per min | length(cm) |
| Cutting system | 0.0316* | 0.0001* | 0.0001* |
| Cutting cycle | 0.2251 | 0.0001* | 0.0150* |
| Cutting system*Cutting cycle | 0.3914 | 0.0001* | 0.0001* |
| (Cutting cycle)² | 0.6701 | 0.2755 | 0.0006* |
| Whole model fit (R²) | 0.172 | 0.622*** | 0.695*** |

TABLE 6
Summary ANOVA from the Oasis ® media volume experiment I

| | Laboratory Prob > F | | |
|---|---|---|---|
| | Multipli- | Plant | The first |
| Terms | cation | cutting rate | three nodes |
| Source of Variation | ratio | per min | length(cm) |
| Media volume mL | 0.0687 | 0.0607 | 0.5227 |
| Cutting cycle | 0.0355* | 0.0001* | 0.0001* |
| Media volume | 0.5700 | 0.3302 | 0.0577 |
| mL*Cutting cycle | | | |
| (Media volume mL)² | 0.8237 | 0.9015 | 0.5558 |
| (Cutting cycle)² | 0.1565 | 0.0740 | 0.0002* |
| Whole model fit (R²) | 0.223 | 0.474** | 0.545*** |

TABLE 7
Summary ANOVA from four cycles of multiple cutting
on agar and Oasis ® using scalpel and forceps
and scissor for Ragtime experiment

| | Laboratory Prob > F | | | |
|---|---|---|---|---|
| | Multipli- | Plant | The first | The shoot |
| Terms | cation | cutting rate | three node | fresh |
| Source of Variation | ratio | per min | length (cm) | mass (g) |
| Cutting system | 0.0001* | 0.0001* | 0.0022* | 0.0001* |
| Cutting cycle | 0.0054* | 0.0050* | 0.5829 | 0.0018* |
| Cutting | 0.0025* | 0.0014* | 0.0001* | 0.0161* |
| system*Cutting | | | | |
| s cycle | | | | |
| (Cutting cycle)² | 0.0134* | 0.0004* | 0.0002* | 0.0044* |
| Whole model fit | 0.344*** | 0.715*** | 0.441*** | 0.420*** |
| (R²) | | | | |

TABLE 8
Summary ANOVA from the Oasis ® media volume experiment II

| | Laboratory Prob > F | | | |
|---|---|---|---|---|
| | Multipli- | Plant | The first | The shoot |
| Terms | cation | cutting rate | three nodes | fresh |
| Source of Variation | ratio | per min | length (cm) | mass (g) |
| Media volume mL | 0.0707 | 0.6800 | 0.1519 | 0.0180* |
| Cutting type | 0.5753 | 0.0001* | 0.4689 | 0.0036* |
| Cutting cycle | 0.4142 | 0.9611 | 0.0002* | 0.0130* |
| Media volume | 0.2860 | 0.4776 | 0.2983 | 0.4155 |
| mL*Cutting type | | | | |
| Media volume | 0.5371 | 0.8805 | 0.6955 | 0.8376 |
| mL*Cutting cycle | | | | |
| Cutting | 0.9455 | 0.9732 | 0.0107* | 0.2507 |
| type*Cutting | | | | |
| cycle | | | | |
| Media volume | 0.5688 | 0.6034 | 0.4769 | 0.9280 |
| mL*Cutting | | | | |
| type*Cutting | | | | |
| cycle | | | | |
| (Cutting cycle)² | 0.0004* | 0.0023* | 0.0001* | 0.0020* |
| (Media volume | 0.0307* | 0.5870 | 0.1105 | 0.8382 |
| mL)² | | | | |
| Whole model fit | 0.301** | 0.674*** | 0.500*** | 0.367** |
| (R²) | | | | |

1. A method of culturing and harvesting plant shoot tips, comprising:
providing a sterile vessel sized and configured to hold at least one plant comprising one or more root masses and a first set of shoot tips;
cutting across a base of the first set of shoot tips with the one or more root masses held in the vessel to cut a first plurality of cut shoot tips at a first time; then
growing a second set of shoot tips from the one or more root masses in the vessel; and then
cutting across the base of the second set of shoot tips with the one or more root masses held in the vessel to cut a second plurality of cut shoot tips at a second time.

2. The method of claim 1, wherein the second set of shoot tips has an increased multiplication ratio providing more shoot tips than that the first set of shoot tips.

3. The method of claim 1, wherein the cutting is carried out using a single direction motion of a cutting tool above and across a base of the vessel.

4. The method of claim 1, wherein the cutting is carried out using a reciprocating motion of a cutting tool above and across a base of the vessel.

5. The method of claim 1, wherein the cutting is carried out using an electric knife.

6. The method of claim 1, wherein the cutting is carried out using a blade that is axially stationary and moved either automatically using a robotic arm or other electromechanical member or moved manually across and above the base.

7. The method of claim 1, wherein the growing of the second set of shoot tips comprises a growth period in a range of about 1 week to about 3 weeks, optionally comprising a growth period in a range of about 1.5 weeks to about 2.5 weeks.

8. The method of claim 1, wherein the first plurality of cut shoot tips and the second plurality of cut shoot tips are collected in a sterile receiver as microcuttings, and wherein the microcuttings are placed in one or more different sterile vessels with nutrients in a greenhouse to grow into full grown plants.

9. The method of claim 1, further comprising:
growing a third set of shoot tips from the one or more root masses held in the vessel after cutting the second set of shoot tips; and
cutting across the base of the third set of shoot tips with the one or more root masses held in the vessel to cut a third plurality of cut shoot tips at a third time.

10. The method of claim 9, further comprising:
growing a fourth set of shoot tips from the one or more root masses held in the vessel after cutting the third set of shoot tips; and then
cutting across the base of the fourth set of shoot tips with the one or more root masses held in the vessel to cut a fourth plurality of cut shoot tips at a fourth time,
wherein at least the base of the vessel remains sterile over each growing step allowing for several harvests of shoot tips from the same root masses, and optionally wherein at least one of the second, third, and fourth set of shoot tips have an increased number of shoot tips per plant relative to the first set of shoot tips.

11. The method of claim 1, further comprising adding nutrients and/or water to the one or more root masses after a respective cutting, wherein the one or more root masses after the cutting of the first plurality of cut shoot tips comprises a rooted matrix with one or more buds thereby allowing for rapid re-growth of one or more additional shoots to yield the second set of shoot tips.

12. The method of claim 1, wherein the vessel has a base releasably attached to a housing, wherein the base has a first height and the housing has a second height, wherein the second height is about 2 times to about 10 times greater than the base, and wherein the base holds the root mass and is sized and configured to allow the first and second set of shoot tips to grow a distance above the base and to be exposed when the housing is detached from the base.

13. The method of claim 1, wherein the vessel has a base with a height that is between about 0.5 inches and about 2 inches, wherein the base has a lateral width that is greater than the height, and wherein the base holds the root mass with the first and second set of shoot tips allowed to grow above the base.

14. The method of claim 13, further comprising a permeable substrate in the base configured to hold the one or more root masses and comprising plant nutrients.

15. The method of claim 13, wherein the base holds a soilless substrate, optionally a plant nutrient and moisture, along with the one or more root masses.

16. The method of claim 13, wherein the base is configured to hold the one or more root masses such that new shoot buds forming the second set of shoot tips after the cutting of the first plurality of cut shoot tips are allowed to grow above the height of the base during the growing step.

17. The method of claim 12, wherein the housing has a sidewall and/or a top comprising one or more permeable membranes.

18. The method of claim 1, wherein the at least one plant is a vascular plant.

19. The method of claim 1, wherein the at least one plant is a gymnosperm or an angiosperm.

20. The method of claim 1, wherein the at least one plant is a dicot or a monocot.

21. The method of claim 1, wherein the at least one plant is cannabis.

22. The method of claim 1, wherein the at least one plant is edible microgreens.

23. The method of claim 1, wherein the at least one plant is a potato.

24. The method of claim 1, wherein the at least one plant is a fruit tree or a timber tree.

25. The method of claim 1, wherein the at least one plant is an ornamental plant.

26. The method of claim 1, wherein the vessel comprises a base, optionally an impermeable rigid or semi-rigid base, and a releasably attached housing, wherein the base is configured to hold the one or more root masses and the first and second sets of shoot tips grow a distance above the base into the housing during the growing steps, and wherein the vessel further comprises a plant support member that is coupled to the base and is configured to allow the first and second sets of shoot tips to grow through and above the plant support member, and wherein the plant support member resides at a height that defines a cut height for the cutting of the first and second sets of shoot tips, optionally wherein the base and/or the housing is visually transmissive.

27. The method of claim 26, wherein the plant support member comprises a thermoformed copolymer.

28. The method of claim 26, wherein an edge of the plant support member forms an interference fit within the vessel.

29. The method of claim 26, wherein the plant support member is concave in shape and applies a spring-like pressure on the shoot tips growing within the vessel such that the shoot tips cannot force the plant support member upward during growth.

30. The method of claim 26, wherein the plant support member comprises a plurality of recesses.

31. The method of claim 26, wherein the plant support member has an open mesh or a grid configuration.

32. The method of claim 31, wherein the mesh or grid configuration comprises laterally spaced apart apertures that have an inverted funnel shape with a larger end residing further away from a bottom of the base to allow increased space for lateral expansion during plant growth.

33. The method of claim 12, the method further comprising removing the housing before the cutting steps and optionally rotating the base from a first orientation to a second orientation between each cutting step.

34. The method of claim 1, wherein the cutting steps are carried out to provide dozens of shoot tips as the first and second set of cut shoot tips.

35. The method of claim 26, further comprising reattaching the housing to the base for the growing of the second set of shoot tips.

36. The method of claim 1, wherein the growing steps are carried out in vitro in a sterile environment with the base maintaining sterility during the growing of the first and second sets shoot tips.

37. The method of claim 1, wherein the first plurality of cut shoot tips and the second plurality of cut shoot tips have an increased rooting percentage and/or an increased percentage of survival in a greenhouse environment compared to at least one standard set of shoot tips collected individually using a scalpel from a corresponding root mass.

38. A plant tissue culture device, comprising:
a sterile base, optionally an impermeable rigid or semi-rigid base; and
a sterile housing releasably attached to the sterile base,
wherein the base has a first height and the housing has a second height, wherein the second height of the housing is about 2 times to about 10 times greater than the first height of the base.

39. The device of claim 38, wherein the base is sized and configured to hold one or more plant root masses therein and allows the one or more plant root masses to grow and produce a set of shoot tips with the set of shoot tips residing in the housing above a top edge of the base, optionally the one or more root masses is held in a matrix of soilless nutrient material.

40. The device of claim 38, wherein the housing comprises one or more permeable membranes.

41. The device of claim 38, further comprising a plant support member that is coupled to the base and configured to allow a set of shoot tips to grow through and above the plant support member, and wherein the plant support member resides at a height that defines a cut height for a cutting of the set of shoot tips.

42. The device of claim 41, wherein the plant support member comprises a thermoformed copolymer.

43. The device of claim 41, wherein an edge of the plant support member forms an interference fit with the base of the device.

44. The device of claim 38, wherein the plant support member is concave in shape and applies a spring-like pressure on the shoot tips growing within the device such that the shoot tips cannot force the plant support member upward during growth.

45. The device of claim 41, wherein the plant support member comprises a plurality of recesses.

46. The device of a claim 41, wherein the plant support member has an open mesh or a grid configuration.

47. The device of claim 46, wherein the mesh or grid configuration comprises a plurality of laterally spaced apart apertures that have an inverted funnel shape with a larger end residing further away from a bottom of the base to allow increased space for lateral expansion during plant growth.
